(19) United States
(12) Patent Application Publication
(10) Pub. No.: US 2022/0369983 A1
(43) Pub. Date: Nov. 24, 2022

(54) SYSTEM AND METHOD FOR 12-LEAD ECG RECORDING AND WIRELESS REMOTE MONITORING

(71) Applicant: Gurunath Purushottam PARALE, Solapur, Maharashtra (IN)

(72) Inventors: Gurunath Purushottam PARALE, Solapur (IN); Shriram Ganesh KULKARNI, Pune (IN); Prashant Govind SADAVARTE, Panvel (IN)

(21) Appl. No.: 17/767,165

(22) PCT Filed: Oct. 5, 2020
(86) PCT No.: PCT/IN2020/050855

§ 371 Date: Apr. 7, 2022

(30) Foreign Application Priority Data
Oct. 11, 2019 (IN) 201921041290

(51) Int. Cl. A61B 5/256; A61B 5/00; A61B 5/11; A61B 5/282; A61B 5/332; A61B 5/333

(52) U.S. Cl. CPC A61B 5/256; A61B 5/0006; A61B 5/0022; A61B 5/1112; A61B 5/282; A61B 5/332; A61B 5/333; A61B 5/6801; A61B 5/6823; A61B 2562/221

(57) ABSTRACT

Disclosed is a system (100) and method (200) for 12-lead ECG recording and wireless remote monitoring. The system (100) provides a wearable ECG acquisition unit (40) recording 12-lead ECG from torso positions of electrode sensors on user's body thereby permitting unrestrained free movement during recording without compromising on accuracy achieved by conventional 12-lead ECG system. Thus, the system (100) and method (200) provides a reliable, quick to deploy, cost effective and portable alternative to bulky conventional 12-lead ECG systems and eliminates need for resorting to skilled physician every time the 12-lead ECG is to be recorded. The system (100) and method (200) is universal in use and torso placement positions, facilitating uniform acquisition of the 12-lead ECG without altering the placement positions of the electrode sensors irrespective of ECG modalities, thereby serving as a standard format for ECG recording and long term ECG monitoring.

### FIELD

The present disclosure relates to electrocardiogram (ECG/EKG) and more particularly provides a system and method for recording and wireless remote transmission and monitoring of 12-lead ECG without resorting to a medical facility.

### BACKGROUND

ECG also called EKG (electrocardiogram) is a graph of electrical activity of heart. ECG analysis is widely used method to study functions and identifying disorders of the heart. Heart attack is number one cause of death in world in general and in India in particular. Around 60% of deaths due to heart attack occur in first hour. ECG analysis is widely used method for diagnosis of a heart attack by recording and analyzing 12-lead ECG indicative of cardiac condition of patient. Further, serial 12-lead ECG monitoring is critical in patients during the early hours of suspected Acute Coronary Syndrome (ACS) when the clinical status and ECG findings frequently change. The current system (i.e. conventional 12-lead ECG system/device) of recording of the 12-lead ECG (i.e. standard ECG) for diagnostic purposes by manually placing electrodes by skilled personnel on chest, wrists and ankles has been in practice for nearly 75 years and is highly accurate in respect of diagnosis of cardiovascular disorders based on the 12-lead ECG obtained therefrom. However, the current system has the following drawbacks:
1. Requirement of manually placing each electrode sensor on the patient increases dependency on the skilled personnel thereby making the process time-consuming, because improper placement of the electrode sensors alters the ECG readings and may lead to false diagnosis and treatment;
2. Placement positions of the electrode sensors on arms and legs restricts mobility thus comfort level of the patient for 12-lead ECG recording is minimal and also demands use of long interconnecting wires/cables to connect arms, legs and chest thereby making the system bulky and non-portable. Thus, the patient is required to pay a visit to hospital every time an ECG is to be recorded which becomes inconvenient to the patient during long term ECG monitoring activity.
3. Recording of different types of ECG/ECG modalities which are available for various cardiac diagnosis (standard 12-lead ECG, stress test ECG or an ambulatory ECG) requires the skilled personnel to manually transpose and attach the electrode sensors to different locations/positions on the patient's body depending upon the ECG modality to ensure the patient's comfort and reduce movement artefacts while ECG recording. As a result, changes in one type of ECG recorded cannot be extrapolated to other type of ECG. Further, these alterations in electrode positions compromises upon the accuracy of the 12-lead ECG which is recorded, thereby misleading diagnosis of certain cardiac conditions.
4. In an out-hospital setting, placement of the electrode sensors on the chest, wrists and ankles becomes cumbersome, moreover, placement of electrodes in distal limb locations is also more prone to motion artefacts.
5. Dependency on the skilled personnel for recording the ECG, requires either the patient to reach the hospital by their own means or paramedics have to reach the patient's location to record the ECG upon onset of symptom. This dependency leads to increase in time from symptom onset to arrival to the healthcare facility, and door to reperfusion time thereby causing significant delays in diagnosis and treatment.

Further, nearly 70% of Indian population resides in rural areas without having any immediate access to the conventional 12-lead ECG device and qualified/skilled personnel to make diagnosis of heart attack. The ECG is the cornerstone for early and accurate diagnosis of acute coronary syndrome (ACS). In ST elevation myocardial infarction (STEMI), the time to revascularization is an established predictor of mortality and infarct size. Total ischemic time (TIT) which is the time from the onset of symptoms to the definitive treatment is well established prognostic determinant of survival in major heat attack (STEMI). TIT constitutes symptom to hospital door time (SDT) and door to treatment (balloon) time (DBT)/door to reperfusion time. Health establishments have successfully made efforts to reduce door to reperfusion time. However, to successfully reduce the time from symptom onset to hospital (SDT), it is crucial to record and interpret the first ECG as early as possible. Today, contemporary telecommunication can easily be exploited to reduce ECG interpretation time, but presently tele-transmission of ECG is restricted to a few leads, mainly to diagnose arrhythmias. For diagnosis of STEMI, either the patient has to reach the hospital by their own means or paramedics have to reach the patient's location to record the first ECG. Both situations involve significant delays in diagnosis and treatment. This delay can be significantly reduced if the patient or relatives could record the first ECG themselves and transmit it to the physician for interpretation. Although these conventional systems have been widely adopted, they are susceptible to poor patient compliance due in part to their bulky form and wired connections to leads. Other portable ECG devices currently available in market addresses few of the afore-mentioned drawbacks, however since these are based only on single or maximum 3 leads (electrode sensors), they work effectively only for rhythm disorder diagnosis and not heart attack, the diagnosis of which essentially requires obtaining the 12-lead ECG from conventional electrode positions on the chest, wrists and ankles of the patient.

Therefore, there exists a need for an alternative 12-lead ECG recording system which is universal in use and electrode positions across the ECG modalities, which is reliable, user-friendly, portable, cost effective and quick to deploy, and which addresses the afore-mentioned drawbacks of the current systems. There is a need to provide an ECG recording system that records the 12-lead ECG with similar accuracy to that obtained by the current 12-lead ECG systems, thereby offering accurate diagnosis of the cardiac abnormalities especially heart attacks. There exists a need to provide an alternative to the conventional electrode positions, the alternative positions which can still facilitate recording of the 12-lead ECG within and even out of hospital environment with ease and no discomfort, allowing unrestricted free movement to the patient while recording the 12-lead ECG without compromising on the accuracy, and which can also significantly reduce the symptom onset to hospital arrival time (SDT) along with DBT. There is a need to widen easy utilization of ECG recording by providing the system that enables lay persons or paramedics to record ECGs at home, ambulance or even in remote inaccessible areas and that allows doctors to remotely monitor, interpret and provide medication to the patient in real-time thus saving precious time and lives in ACS and other cardiac disorders, and also avoiding unnecessary hospital visits.

### OBJECTS

It is an object of the present invention to provide a system for 12-lead ECG recording and wireless remote monitoring.

It is another object of the present invention to provide a method for 12-lead ECG recording and wireless remote monitoring.

It is another object of the present invention to provide a wearable ECG assembly (or unit) and system and method that is light weight, portable, cost effective, user friendly and reliable for recording of standard 12-lead ECG and wireless remote monitoring thereof of a patient.

It is still another object of the present invention to provide a wearable ECG assembly and system and method which integrates therewithin communication means to allow automatic or manual and remote communication capabilities between said wearable ECG assembly and healthcare personnel, hospital or any other monitoring facility in real-time.

It is yet another object of the present invention to provide a wearable ECG assembly and system and method which is universal, and requires no alteration of electrode positions irrespective of the type of ECG to be recorded, thereby eliminating dependency upon the skilled personnel and avoiding unnecessary hospital visits of the patient for each instance the 12-lead ECG is to be recorded.

### SUMMARY OF THE INVENTION

The present invention provides a system and method for 12-lead ECG recording and wireless remote monitoring. The system and the method records the 12-lead ECG that is equivalent to a standard 12-lead ECG which is recorded using a conventional 12-lead ECG system and from conventionally advocated placement positions of 10 electrode sensors thereof. Accordingly, the system of the present invention comprises of a wearable ECG acquisition unit, a plurality of communication device and a communication module. The afore-mentioned components are operably connected with each other to facilitate recording and wireless remote monitoring of cardiac activity of a user/patient.

The wearable ECG acquisition unit facilitates acquisition of 12-lead ECG signals and thereafter generation of the 12-lead ECG therefrom subsequent to wearing of the wearable ECG acquisition unit by the user on torso region thereof. The wearable ECG acquisition unit comprises of a support substrate, a first component preconfigured on the support substrate and a second component electrically coupled to the first component. The support substrate forms body of the wearable ECG acquisition unit to facilitate mounting/configuration of the first component thereacross. The first component is an electrode sensor assembly being preconfigured and fixedly positioned across the support substrate for adhering at positions specifically on the torso region of the user to collectively acquire 12-lead ECG signals therefrom when the wearable ECG acquisition unit is worn by the user. The electrode sensor assembly comprises of 10 electrode sensors including, six precordial electrode sensors (V1-V6) and four limb electrode sensors (RA, LA, RL, LL). The six precordial electrode sensors (V1-V6) adhere to their conventionally advocated placement positions on chest region of the user to acquire precordial ECG signals therefrom when the wearable ECG acquisition unit is worn. That is, to elaborate, the six precordial electrode sensors (V1-V6) are preconfigured across the support substrate for adhering to skin of the user at said positions in a manner such that when the wearable ECG acquisition unit is worn, amongst the six precordial electrode sensors (V1-V6), V1 and V2 gets disposed to lie on fourth intercostal space on opposite side of sternum and equidistant therefrom, V4 gets disposed to lie on fifth intercostal space at mid-clavicular line of the user, V3 gets disposed to lie midway between placement position of V2 and V4, whereas V5 and V6 gets disposed to lie on the fifth intercostal space at anterior-axillary line and mid-axillary line respectively to acquire the precordial ECG signals from these conventional placement positions on the user, wherein, placement positions of the V4, V5 and V6 generally define a horizontal plane. The four limb electrode sensors (RA, LA, RL, LL) adheres to their proposed positions on the chest (near shoulder) and abdominal region of the user to acquire ECG signals therefrom when the wearable ECG acquisition unit is worn. That is, to elaborate, the four limb electrode sensors (RA, LA, RL, LL) are preconfigured across the support substrate for adhering to skin of the user at said proposed positions in a manner such that when the wearable ECG acquisition unit is worn, amongst the four limb electrode sensors (RA, LA, RL, LL), RA and LA gets disposed to lie exactly in or adjacent to right and left delto-pectoral groove of the user below lateral end of clavicle respectively, whereas RL and LL gets disposed to lie at a distance ranging from 1-3 inches above and left-lateral to umbilicus of the user at 12 o'clock and 3 o'clock position respectively. The electrode sensor assembly thus facilitates acquisition of the 12-lead ECG signals from the torso region of the user when the wearable ECG acquisition unit is worn. The electrode sensor assembly of the wearable ECG acquisition unit is additionally is provided with a global positioning system (GPS) to provide location data of the user facilitating allocation of nearest possible healthcare provider to the user for receiving immediate medical assistance. The second component is a signal processing unit disposed in electrical communication (or is electrically coupled) to the first component for receiving the 12-lead ECG signals therefrom, and thereafter facilitating processing thereof to generate the 12-lead ECG therefrom which indicates the cardiovascular activity/health of the user. The signal processing unit is adapted with the capability of wireless transmission of the 12-lead ECG to the user and/or to any other external computing device to facilitate wireless remote monitoring of the cardiac condition of the user.

The plurality of communication device is operatively coupled to the second component of the wearable ECG acquisition unit for receiving the 12-lead ECG transmitted wirelessly therefrom. Each of the plurality of communication device essentially represents a computing device and is configured with a web application for enabling real-time interaction and remote exchange of information/data therebetween. Particularly, the system includes a plurality of users and healthcare providers (for example, doctor) each being associated with the communication device independently coupled thereto, wherein the web application preconfigured thereon presents the information received from the signal processing unit and further facilitates audio-visual communication between the user and the healthcare provider over a communication network thereby enabling wireless remote monitoring of the cardiovascular health of the user by the healthcare provider even though the user is located remote thereto.

The communication module is operatively coupled to the components of the system for establishing the communication network, and preferably a wireless communication network to allow flow of information therebetween. Specifically, the communication module is coupled to the second component and each of the plurality of communication device to enable interaction therewithin and therebetween for transmission of the 12-lead ECG and data related thereto, and to facilitate interaction between the user and the healthcare provider to share data and receive medication therefrom thereby facilitating effective real-time monitoring and management of the cardiovascular health of the user by the healthcare provider located remote thereto.

Thus, the system provides for the wearable ECG acquisition unit which is user-friendly and can be deployed for recording of varied ECG's without changing the placement positions of the electrode sensors on the user's body, thereby eliminating need of a skilled physician to perform ECG recordings. Particularly, adherence positions of the four limb electrode sensors (RA, LA, RL, LL) on the torso only region of the user, imparts portability so that the user can take ECG recordings at comfort of their home without interference in routine activities. Adherence of the four limb electrode sensors (RA, LA, RL, LL) at the proposed fixed torso only positions on the user facilitates uniform acquisition and recording of the 12-lead ECG across varied ECG modalities without changing the adherence positions, thereby making the wearable ECG acquisition unit universal in use irrespective of the type of ECG (i.e. ECG modality) is to be recorded. This also allows for extrapolation of ECG changes across the ECG modalities which includes recording of resting, ambulatory and stress ECG and continuous monitoring of ECG during ongoing surgical procedures in ICU and telemetry. Thus, the wearable ECG acquisition unit provides for a cheaper and a user friendly alternative to the conventional 12-lead ECG system, without compromising on accuracy achieved therefrom i.e. the wearable ECG acquisition unit provided by the system of the present invention facilitates recording of the 12-lead ECG that is equivalent to the standard 12-lead ECG (i.e. ECG recorded from the conventionally advocated placement positions of the electrode sensors on the chest and limbs) and thus aids in diagnosis of cardiovascular disorders and heart attacks. In addition, the wearable ECG acquisition unit permits unconstrained free movement during monitoring and the wearer is unaware of the presence of the electrode assembly thereby facilitating a long term wear and versatility and with ease and comfort.

The present invention, in another aspect, provides the method for 12-lead ECG recording and wireless remote monitoring. The method involves the user wearing the wearable ECG acquisition unit provided by the system of the present invention subsequent to which the electrode sensor assembly gets placed at respective positions only on the torso region of the user to capture the 12-lead ECG signals therefrom. Thereafter, the method involves processing of the 12-lead ECG signals by the signal processing unit to generate the 12-lead ECG further to which it is wirelessly transmitted therefrom to the communication device of the user, and thereafter which the web application executing on the communication device automatically allocates and sends the data to the communication device of the healthcare provider located remote for interpretation and receiving medication therefrom in real-time over the communication network. The user and the healthcare provider interacts in real-time using the web application executing on the communication device independently coupled thereto thereby facilitating wireless remote monitoring of the cardiovascular health of the user by the healthcare provider.

Hence, the system is a cost effective, portable and versatile alternative to the conventional bulky 12-lead ECG systems deployed in hospitals without compromising on the accuracy when compared therewith. The system and the method facilitates remote monitoring of the patient by the doctor and ensures that the patient receives immediate medical assistance when demanded without any delay when the patient is sitting at comfort of their home. The present invention thus enables rapid diagnosis of the cardiovascular condition in remote areas in underdeveloped countries and also reduces symptom to door time in developed countries thus saving many lives.

### BRIEF DESCRIPTION OF DRAWINGS

The objectives and advantages of the present invention will be more clearly understood from the following description of the invention taken in conjunction with the accompanying non-limiting drawings, wherein:

FIG. 1 is a schematic front view of human showing conventional electrode sensor placement positions used for obtaining a standard 12-lead ECG (resting 10 electrode 12-lead ECG);

FIG. 2 shows an overall representation (block diagram) of components of the system for 12-lead ECG recording and wireless remote monitoring, in accordance with the present invention;

FIGS. 3 and 4 is a schematic front view of human torso showing the proposed torso only electrode sensor placement positions used for obtaining the 12-lead ECG, wherein six precordial electrode sensors are seen conventionally placed whereas four limb electrode sensors are placed at proposed torso positions according to the present invention;

FIGS. 5 and 6 illustrates some example implementations of the wearable ECG acquisition unit of the system embodying the invention, in accordance with the present invention;

FIG. 7 shows a flow-chart of the method for 12-lead ECG recording and wireless remote monitoring, in accordance with the present invention;

FIGS. 8-10 shows graphical representations of comparison between the conventional ECG (c-ECG) and the torso ECG (t-ECG) in different subsets of ACS, in accordance with a sample case study run for validating the proposed torso only electrode positions of the present invention for accurate diagnosis of ACS; and

FIG. 11 shows angles of Einthoven triangle formed in various limb lead placement systems, wherein,

FIG. 11 (a) shows angle formed between the limb electrode sensor positions in the conventional limb lead placement system,

FIG. 11 (b) shows angle formed between the limb electrode sensor positions in the torso limb lead placement system proposed by the present invention,

FIG. 11 (c) shows angle formed between the limb electrode sensor positions in the Mason-Likar limb lead placement system.

### DETAILED DESCRIPTION

The present invention provides a system and method for 12-lead ECG recording and wireless remote monitoring. The system and the method facilitates recording of the 12-lead ECG having accuracy equivalent to that recorded using a conventional 12-lead ECG system deployed in medical facilities. The system and the method facilitates recording of the resting, ambulatory and stress ECG using fixed placement positions of electrodes/electrode sensors on user thereby serving as a universal system and method for uniform recording of the 12-lead ECG. Further, portability of the system facilitates real-time recording of the 12-lead ECG and wireless remote monitoring of cardiovascular health of the user/patient from comfort of their home without imposing any mobility restrictions thereon and without resorting to any medical facility.

The features, functioning, arrangement and components of the system and conditions of operation and steps of the method of the present invention include but are not limited to the disclosure provided herein below. The methods or processes, the sequence and/or arrangement of steps described herein are illustrative and not restrictive. It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

This present invention is illustrated with reference to the accompanying drawings, throughout which reference numbers/labels indicate corresponding parts in the various figures. These reference numbers are shown in bracket in the following description.

In accordance with one aspect of the present invention, a system (100), hereinafter referred to as “the system (100)”, for 12-lead ECG recording and wireless remote monitoring is disclosed. In accordance with another aspect of the present invention, a method (200), hereinafter referred to as “the method (200)”, for 12-lead ECG recording and wireless remote monitoring is disclosed. In an embodiment, continuous monitoring and simultaneous availability of the 12-lead ECG and cardiac output of a patient, using a single wearable unit-system is disclosed herein below. The system (100) and the method (200) facilitates recording of the 12-lead ECG (electrocardiogram/EKG) which is identical/equivalent to a standard 12-lead ECG. The system (100) and the method (200) also facilitates wireless remote transmission of the 12-lead ECG of a user to a healthcare provider thereby enabling real-time wireless remote monitoring and management of cardiovascular health of the user. The system (100) and the method (200) proposes a wearable appliance/unit (or component and like) that is to be worn by the user for recording of the 12-lead ECG thereof even while performing daily activities without discomfort.

### Definitions

The term “wearable” for the purpose of present disclosure and without departing from the dictionary meaning thereof refers to any component/appliance/unit (or body worn device) that is small and light enough to be detachably worn (for example, like a garment), or placed/applied (for example, on wrist etc.) or carried (attached) on any body part of the user without offering interference in carrying out day to day activities to the user. The wearable unit is a unit that is directly, but not permanently affixed to the user's body using any adhesive or non-adhesive means.

The term “equivalent” for the purpose of present disclosure and without departing from the dictionary meaning thereof refers to measurement/comparison in terms of accuracy in diagnosis of cardiovascular disorders including heart attacks. That is, equivalency does not simply imply comparison of ECG waveforms of the 12-lead ECG obtained using the present and the conventional system, but rather is quantified based on the accuracy in diagnosis of the cardiovascular disorders from the 12-lead ECG obtained using the present system when compared with that from the standard 12-lead ECG obtained using the conventional 12-lead ECG system.

For the purpose of present disclosure, the term “standard 12-lead ECG/EKG” refers to a standard/resting 10 electrode 12-lead ECG (or a resting ECG) acquired using a conventional 12-lead ECG system wherein ECG readings are taken from conventionally advocated placement positions of electrode sensors (or electrodes) on chest and limb region of a user.

For the purpose of present disclosure, the term “conventional 12-lead ECG system” refers to conventionally used bulky devices/apparatus/machines/systems in medical and like facilities (for e.g. hospital) for recording (or acquisition) of the 12-lead ECG (i.e. the standard 12-lead ECG), wherein, the conventional 12-lead ECG system consists of 10 electrode sensors including six precordial and four limb electrode sensors and an ECG measurement device/monitor electrically coupled to the 10 electrode sensors, wherein, the 10 electrode sensors acquires and transmits analogue ECG signals when placed manually by a skilled physician at the conventionally advocated placement positions thereof on skin of the user to an ECG monitor which then performs digitization of the analogue ECG signals to generate the 12-lead ECG (i.e. the standard 12-lead ECG) of the user.

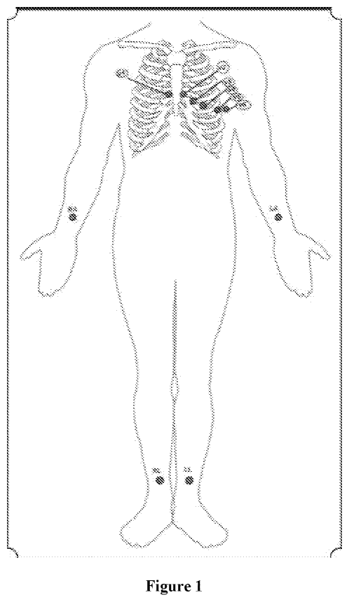

For the purpose of present disclosure, the term “conventionally advocated placement positions” refers to conventional (or standard) placement positions (i.e. electrode configuration) of the 10 electrode sensors on the chest and limb region of the user from where the analogue ECG signals (i.e. raw ECG signals in analogue format) are sensed and recorded to obtain the standard 12-lead ECG therefrom. The conventional placement positions of the 10 electrode sensors as shown in FIG. 1, wherein each electrode sensor is depicted/referred hereinafter using corresponding label, is as follows:

For Six precordial/chest electrode sensors (V1-V6), the conventional placement positions on the chest region of the user for acquiring precordial ECG signals therefrom is as follows:

V1—Fourth intercostal space on the right side of sternum

V2—Fourth intercostal space at the left side of sternum

V3—Midway between placement of V2 and V4

V4—Fifth intercostal space at the mid-clavicular line

V5—Anterior-axillary line on the same horizontal level as V4

V6—Mid-axillary line on the same horizontal level as V4 and V5

For Four limb electrode sensors (Right arm (RA), Left arm (LA), Right leg (RL) and Left leg (LL)), the conventional placement positions on the limb region of the user for acquiring ECG signals therefrom is as follows:

RA (Right Arm)—On right wrist

LA (Left Arm)—On left wrist

RL (Right Leg)—On right ankle

LL (Left Leg)—On left ankle

For the purpose of present disclosure, the term “healthcare provider” refers to any medical or health practitioner and/or healthcare facilities that offers medical care to individuals, and includes, but is not limited thereto, a central healthcare facility (for example: a hospital), a medical professional/assistant, a doctor, user's caregiver, a paramedic, EMT, medical staff, a clinician, a physician/technician and like personnel's and/or healthcare facilities having known medical capabilities and certifications for acquisition, interpretation and administration of treatment/medication to the individual based on analysis of the 12-lead ECG obtained therefrom.

For the purpose of present disclosure, the term “cardiovascular health” refers to cardiovascular events/activity and conditions of the individual and includes, but is not limited thereto, cardiac/heart condition, activity, cardiac status and like conditions.

For the purpose of present disclosure, the term “user” refers to any personnel/individual (for example: a patient) using the system (100) of the present disclosure for recording and wireless transmission of the 12-lead ECG thereby facilitating real-time wireless remote monitoring of the cardiovascular health by the healthcare provider to receive immediate treatment/medication therefrom.

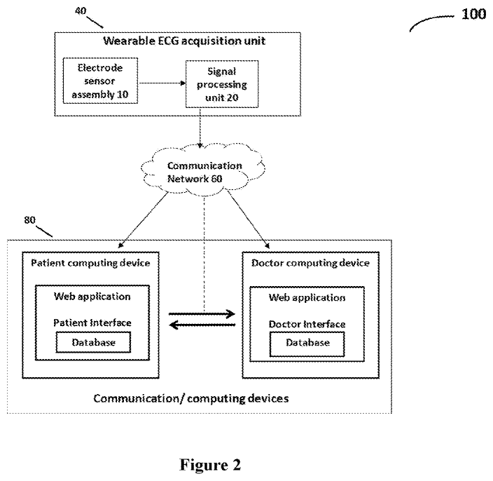

Accordingly, referring to FIG. 2, the system (100) comprises of, not limiting to, a wearable ECG acquisition unit (40), a plurality of communication device (80) and a communication module. The afore-mentioned individual components/parts of the system (100) are operably coupled to each other for recording of the 12-lead ECG and wireless transmission thereof to the healthcare provider to facilitate wireless remote monitoring of the cardiovascular health of the user.

The wearable ECG acquisition unit (40) is a wearable component of the system (100) and is adapted to acquire the 12-lead ECG of the user when worn around torso region thereof. The wearable ECG acquisition unit (40) comprises of a support substrate, and disposed thereacross a first component operably and electrically coupled to a second component.

The support substrate is a supporting matrix/material forming body of the wearable ECG acquisition unit (40) that is operable to attach/couple to the user.

The first component is an electrode sensor assembly (10) preconfigured and fixedly positioned (at predetermined location) across (or at discrete locations on) the support substrate for adhering at positions on the torso region of the user to acquire 12-lead ECG signals therefrom when the wearable ECG acquisition unit (40) is worn by the user. In an alternate embodiment, the electrode sensor assembly (10) is detachably disposed at fixed positions across the support substrate allowing addition of other sensors, if required. The electrode sensor assembly (10) comprises of 10 electrode sensors (i.e. ECG sensors), specifically, six precordial electrode sensors (V1-V6) and four limb electrode sensors (RA, LA, RL, LL) which are distributed to predispose (or preconfigured/aligned) at fixed predetermined positions across the support substrate such that they adhere to their respective proposed positions on the torso region (or trunk region) to acquire the 12-lead ECG signals therefrom when the wearable ECG acquisition unit (40) is worn by the user. The 12-lead ECG signals post-processing and transformation generates the 12-lead ECG of the user. In an embodiment, the electrode sensor assembly (10) is preconfigured across an inner surface of the support substrate facing towards the skin of the wearer. In another embodiment, the electrode sensor assembly (10) is preconfigured across an outer surface opposite to the inner surface of the support substrate orienting towards external environment and facing away from the skin of the wearer. Here, the term ‘wearer’ refers to the user who is wearing the wearable ECG acquisition unit (40) as provided by the system (100) of the present invention. Further, the term ‘12-lead ECG signals’ for the purpose of present disclosure, refers to ECG signals/waveform (or ECG voltages) acquired collectively by the electrode sensor assembly (10) (i.e. the ECG signals sensed by the 10 electrode sensors through 12 different angles) from the chest and the abdominal region when the wearable ECG acquisition unit (40) is worn by the user, wherein, the ECG signals are present in raw/unprocessed, analogue, non-readable and non-transmittable format. Furthermore, the term ‘12-lead ECG’, for the purpose of present disclosure, refers to digitized ECG signals generated post-processing of the 12-lead ECG signals, wherein, the 12-lead ECG is represented in graphical, human-readable and wirelessly transmittable format.

The electrode sensor assembly (10) is preconfigured and fixedly positioned across the support substrate such that upon wearing of the wearable ECG acquisition unit (40) by the user, the 10 electrode sensors of the electrode sensor assembly (10) disposes to position themselves and adhere to their respective proposed positions on chest and abdominal region of the user for sensing the ECG signals therefrom through 12 different angles (i.e. acquire the 12-lead ECG signals). In an embodiment, the electrode sensor assembly (10) is preconfigured (or positioned) across the support substrate by way of mounting/attachment at fixed predetermined positions thereon such that the 10 electrode sensors appear protruding outwards from the inner surface of the support substrate to establish close direct contact with skin of the wearer for acquisition of the 12-lead ECG signals therefrom. In another embodiment, the electrode sensor assembly (10) is preconfigured across the support substrate by way of integration at fixed predetermined positions therewithin such that the 10 electrode sensors lie integrated/embedded (or interlaced) within the support substrate to establish concomitant contact with the skin surface of the wearer at respective proposed positions thereon for acquisition of the 12-lead ECG signals therefrom when the wearable ECG acquisition unit (40) is worn. In a preferred embodiment, the electrode sensor assembly (10) is so preconfigured across the support substrate such that the 10 electrode sensors get automatically disposed to lie (or gets placed) at their respective proposed positions on the chest and the abdominal region to acquire the ECG signals therefrom when the wearable ECG acquisition unit (40) is worn by the user. In short, the 10 electrode sensors are so preconfigured and distributed across the support substrate such that when the user wear's the wearable ECG acquisition unit (40), the 10 electrode sensors gets automatically placed/disposed to lie (and/or adhere) at said positions on the user for acquisition of the 12-lead ECG signals therefrom, thereby eliminating need for manual attachment of the electrode sensor at said positions with help of a skilled physician. In an alternate embodiment, however, the 10 electrode sensors of the electrode sensor assembly (10) are manually placed and directly attached using extendable wires/electrical connections coupled thereto at said positions by the wearer, wherein the 10 electrode sensors acquire voltage signals therefrom through these connected wires to facilitate acquisition of the 12-lead ECG signals. However, not limiting thereto, it is evident that a combination of above embodiments can be deployed to facilitate acquisition of the 12-lead ECG signals by the electrode sensor assembly (10). For example, the electrode sensor assembly (10) is so placed and preconfigured across the support substrate such that when the wearable ECG acquisition unit (40) is worn by the user, the six precordial electrode sensors (V1-V6) gets automatically placed/disposed to lie and/or adhere at their respective positions on the chest region for acquiring precordial ECG signals therefrom whereas, the four limb electrode sensors (RA, LA, RL, LL) requires manual attachment at their respective proposed positions on the chest (near shoulder region) and the abdominal region of the wearer to facilitate acquisition of the ECG signals therefrom.

The adherence of the 10 electrode sensors to their respective positions on the skin of the wearer is through direct contact/attachment or via indirect contact, and can be achieved through numerous techniques/ways known in the art that are capable of placing and retaining the electrode sensor assembly (10) securely against the skin of the wearer to facilitate acquisition of quality ECG signals therefrom. For example, the 10 electrode sensors with high friction surfaces may be used to minimize slippage and allow them to stay in place on the skin of the wearer, or in another example, the 10 electrode sensors may be coated with slightly sticky surfaces/adhesive/fixing agent which allows direct contact and holds them in place against the skin of the wearer. In an embodiment, adherence of the electrode sensor assembly (10) at said positions on the wearer is achieved through mechanisms which avoids use of any adhesives/fixing agents to maintain a fairly continuous physical contact with the skin of the user. The need for deployment of these mechanisms might arise when the electrode sensor assembly (10) is preconfigured by way of integration/interlacing within the support substrate such that it gets automatically placed/disposed (as opposed to manual placement/direct contact of the electrode sensors) at respective positions when the wearable ECG acquisition unit (40) is worn by the user for acquiring the ECG signals therefrom and thus requires establishment of a tight contact with the skin surface of the wearer to record accurate and quality ECG signals therefrom, or wherein the electrode sensor assembly (10) senses the 12-lead ECG signals by establishing indirect contact with the skin of the user. Particularly, the mechanisms that hold the 10 electrode sensors against the skin of the wearer includes a tensile/compressive force (or pressure) imparted on the electrode sensor assembly (10) either internally or externally to maintain/secure a continuous physical contact of the 10 electrode sensors with the skin of the wearer at said positions thereon to achieve accurate recording of the 12-lead ECG signals therefrom throughout the monitoring period. For example, additional structural supports such as elastic bands, inflatable structures etc. may be deployed to be worn above the wearable ECG acquisition unit (40) for exertion of external force, or alternatively may be provided in the form of an internal support structure to exert a compressive force on the electrode sensor assembly (10) sufficient to hold it firmly (i.e. by establishing a tight physical contact) against the skin of the wearer and in continuous contact therewith throughout the monitoring period, since loose contacts there between affects quality of the 12-lead ECG signals obtained therefrom. In an embodiment, the internal structures of the wearable ECG acquisition unit (40) may be biased to press snugly against the skin in at least those portions of the wearable ECG acquisition unit (40) where the electrode sensor assembly (10) is needed to be held in a relatively stable orientation. Further, in another embodiment, the compressive forces are imparted upon the electrode sensor assembly (10) such that the electrode sensor assembly (10) gets firmly pinned in place against the skin of the wearer, although not directly adhered (i.e. direct attachment) but sufficient enough to sense the ECG signals therefrom. However, it is evident to a person skilled in the art that any other type of fastening/attachment/adherence mechanism or any other technique may be used to closely hold the electrode sensor assembly (10) in place against the skin of the wearer at said positions on the chest and the abdominal region thereof to facilitate accurate acquisition of the 12-lead ECG signals therefrom. It is further evident that the wearable ECG acquisition unit (40) is required to keep the electrode sensor assembly (10) oriented on the skin at said positions thereon during monitoring, but the electrode sensor assembly (10) need not be in continuous and stationary contact with the skin of the wearer at all times and some degree of sliding movement along the skin surface is permissible as long as at least a part of the electrode sensor's surface contacts the skin for acquisition of the 12-lead ECG signals therefrom.

10 Electrode Sensors of the Electrode Sensor Assembly (10)

The six precordial electrode sensors (V1-V6) of the electrode sensor assembly (10) are adapted to adhere, sense and acquire precordial ECG signals (or ECG voltages) from the chest region of the user subsequent to wearing of the wearable ECG acquisition unit (40) on/around the torso region thereof. In a preferred embodiment of the present invention, the six precordial electrode sensors (V1-V6) are preconfigured at fixed predetermined positions across (on or within) the support substrate such that upon wearing of the wearable ECG acquisition unit (40) by the user, the six precordial electrode sensors (V1-V6) gets positioned/placed/disposed to lie and adhere to their respective positions (i.e. precordial points) on the chest region of the user to acquire the precordial ECG signals therefrom. Particularly, in accordance with the present invention, subsequent to wearing of the wearable ECG acquisition unit (40) by the user, the precordial points on the chest region of the wearer where the six precordial electrode sensors (V1-V6) gets placed and adheres to, coincides exactly with the conventionally advocated placement positions (i.e. the conventional placement positions) thereof, cited as follows:
V1 and V2 gets disposed to lie at fourth intercostal space on opposite side of sternum and equidistant therefrom, V1 disposes on right side of the sternum whereas V2 disposes on left side of the sternum,
V4 gets disposed to lie at fifth intercostal space at mid-clavicular line of the user,
V3 gets disposed to lie equidistant from/midway between placement position of V2 and V4,
V5 gets disposed to lie at the fifth intercostal space at anterior-axillary line, and
V6 gets disposed to lie at the fifth intercostal space at mid-axillary line, wherein, positions of the V4, V5 and V6 generally define a horizontal plane.

In short, the six precordial electrode sensors (V1-V6) are preconfigured across the support substrate such that they get placed to adhere at positions overlapping with their conventionally advocated placement positions on the chest region subsequent to wearing of the wearable ECG acquisition unit (40) by the user. Therefore, the precordial ECG signals acquired by the six precordial electrode sensors (V1-V6) of the present invention from their conventional placement positions on the chest region are identical (or equivalent) to those obtained by using the conventional 12-lead ECG system, and thus offers identical accuracy when compared therewith.

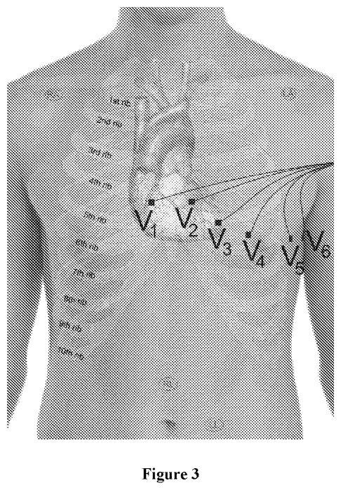

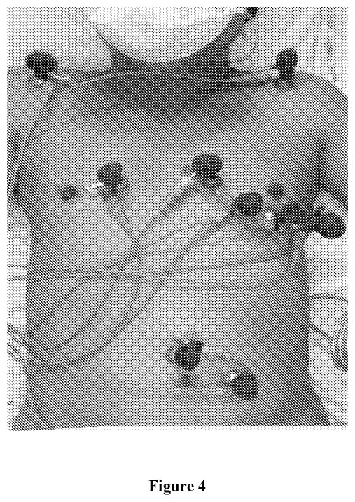

The four limb electrode sensors (RA, LA, RL, LL) of the electrode sensor assembly (10) are adapted to adhere, sense and acquire ECG signals (or ECG voltages i.e. raw ECG signals) from the chest and the abdominal region of the user subsequent to wearing of the wearable ECG acquisition unit (40) on/around the torso region thereof. In a preferred embodiment of the present invention, the four limb electrode sensors (RA, LA, RL, LL) are preconfigured at fixed predetermined positions across (on or within) the support substrate such that upon wearing of the wearable ECG acquisition unit (40) by the user, the four limb electrode sensors (RA, LA, RL, LL) gets positioned/placed/disposed to lie and adhere to their respective positions on the chest (specifically near shoulder, i.e. collar bone) and the abdominal region (near umbilicus) of the user to acquire the ECG signals therefrom. Particularly, in furtherance to afore-stated statement and in accordance with the present invention, subsequent to wearing of the wearable ECG acquisition unit (40), the said positions on the chest and the abdominal region where the four limb electrode sensors (RA, LA, RL, LL) disposes and adheres on the skin of the wearer as shown in FIGS. 3 and 4 are as follows:
RA gets disposed to lie exactly in or adjacent to right delto-pectoral groove below lateral end of clavicle of the user,
LA gets disposed to lie exactly in or adjacent to left delto-pectoral groove below the lateral end of the clavicle of the user,
RL gets disposed to lie at an approximate distance of, not limited thereto, 1-3 inches above umbilicus and representing 12 o'clock position with respect to the umbilicus of the user, and
LL gets disposed to lie at an approximate distance of, not limited thereto, 1-3 inches left lateral to the umbilicus and representing 3 o'clock position with respect to the umbilicus of the user.

In short, according to the present invention, the four limb electrode sensors (RA, LA, RL, LL) are preconfigured across the support substrate such that they get placed to adhere at torso electrode positions which are different than their conventionally advocated placement positions on the limb region subsequent to wearing of the wearable ECG acquisition unit (40) by the user. However, the ECG signals acquired by the four limb electrode sensors (RA, LA, RL, LL) from these proposed torso electrode positions are exactly identical (or equivalent) to those obtained from their conventional advocated placement positions on the user thereby offering accuracy identical when compared therewith. Therefore, although the four limb electrode sensors (RA, LA, RL, LL) are placed and adhered at said positions proposed (i.e. the torso electrode positions) by the present invention which seem to be different than the conventional placement positions thereof, the ECG signals recorded from these proposed positions are equivalent in accuracy as compared to those recorded from the conventional placement positions of the four limb electrode sensors (RA, LA, RL, LL).

Therefore, the electrode sensor assembly (10) facilitates capturing of the 12-lead ECG signals sensed collectively by the 10 electrode sensors through 12 different lead or angles and from the proposed positions on the chest and the abdominal region with accuracy comparable to the conventional 12-lead ECG system and without imposing mobility restrictions on the wearer due to the modified positions of the four limb electrode sensors (RA, LA, RL, LL). In an embodiment, acquisition of the 12-lead ECG signals is in real-time and can be continuous using a power source (for example, a rechargeable battery, AC supply and like) or can be at regular intervals (for example, every 10 secs) or on-demand when faced with symptomatic complains by the user. The ECG sensor data captured by the electrode sensor assembly (10) is thereafter fed to the second component for processing and analysis. In an alternate embodiment, the wearable ECG acquisition unit (40) additionally includes a positioning system or specifically a global positioning system (GPS) module/receiver that relays further the position/location co-ordinates of the wearer each time along with the ECG data collected by the electrode sensor assembly (10) to the second component to offer mobility tracking thereby facilitating reception of immediate medical assistance from the healthcare provider located remote to the user at any instance. Additionally, the positioning system may be used to locate the user/wearer during an emergency situation.

The second component is a signal processing unit (20) operably/operatively and electrically coupled (i.e. physically) to the first component (i.e. the electrode sensor assembly (10)) for receiving the 12-lead ECG signals (raw signals) and generating the 12-lead ECG (processed signals) therefrom. Particularly, the electrode sensor assembly (10) is provided with a plurality of electrical connections/contacts through which the signal processing unit (20) receives and records the 12-lead ECG. Specifically, one end of each of the electrical connection is connected to one of the electrode sensor and is terminated at the other end to connect with the signal processing unit (20). Thus, the electrode sensor assembly (10) (i.e. the 10 electrode sensors) electrically interfaces with the signal processing unit (20) through the electrical connections thereby transmitting the 12-lead ECG signals there through to facilitate recording (i.e. generation) of the 12-lead ECG therefrom. In an alternate non-limiting embodiment, however, the electrical connections can be adapted to wirelessly interface with a wireless transceiver over which the 12-lead ECG signals sensed by the electrode sensor assembly (10) are transmitted to the signal processing unit (20) coupled wirelessly to the wireless transceiver. Along with the 12-lead ECG signals, location information/signals is also received by the signal processing unit (20) from the electrode sensor assembly (10). The signal processing unit (20), in one embodiment, forms an integral part of the wearable ECG acquisition unit (40) and is disposed resident thereto (on or within the support substrate) in physical connection with the electrode sensor assembly (10) thereof. In another embodiment, the signal processing unit (20) is externally coupled (provided as a standalone unit) to the wearable ECG acquisition unit (40) and is disposed remotely thereto (i.e. disposed proximate or remote from the electrode sensor assembly (10)) but in electrical connection with the electrode sensor assembly (10) thereof. The signal processing unit (20) contains electronic circuitry for recording (i.e. generation of the 12-lead ECG), storing the wearer's electrocardiography as sensed via the electrode sensor assembly (10) and transmitting the 12-lead ECG (and other data related thereto including the location co-coordinates of the user received from the GPS module) further to facilitate wireless remote monitoring of the user. Thus, the signal processing unit (20) essentially includes components, such as but not limiting thereto, an amplifying module, a processing module, a signal conditioning module, a microcontroller, an analogue to digital converter, a storage module, an analysing module, a transmitting/transmission module and like for facilitating performance of its core functions of recording, storage and transmission of the 12-lead ECG and location data over the communication network (60) thereby facilitating immediate diagnosis to provide treatment to the user. In a non-limiting embodiment, the signal processing unit (20) is additionally coupled to other components such as, not limiting to, an external output unit (for example, a display, alarm, LED etc.) for presenting the processed signal and/or information extracted from the processed signal during or after processing, to facilitate real-time viewing of the information in in form of analysis report and the 12-lead ECG of the user in graphical representative format, and may also additionally have a printout facility coupled thereto. Further, the transmission module is preferably a wireless transmission module establishing a wireless communication network (60) facilitating the signal processing unit (20) to wirelessly transmit the 12-lead ECG and location information therefrom (i.e. transmitting the processed signal and the information extracted from the processed signal) to an external storage device (for example, a central server), a network, and/or a computing device or any other like devices capable of wireless communication (for example, smartwatches) to facilitate wireless remote monitoring of the cardiovascular health of the user. The signal processing unit (20) communicates with the external storage device via a web application executing on the external storage/computing device. In a non-limiting example embodiment, the transmission module transmits the 12-lead ECG and information related thereto (including, for example, the location co-ordinates of the wearer) via wireless networks, radio frequency communication protocols, Bluetooth, near-field communication (NFC), and/or optically using infrared or non-infrared LEDs. In a further embodiment, the signal processing unit (20) may contain additional components and functionalities, and may be deployed for monitoring other types of physiology, in addition to ECG, with assistance of other types of sensors. The signal processing unit (20) facilitates quantification or analysis of the data indicative of ECG measurement to provide an indication of the cardiac activity/condition of the user. Particularly, the signal processing unit (20) is configured to perform clean-up and processing of the 12-lead ECG signals using techniques known in the art to generate the 12-lead ECG therefrom. The signal processing unit (20) performs processing to generate electrical graph of the 12-lead ECG, by steps including but not limited thereto, computation of voltage (or potential) differences amongst different points on the skin of the user, amplification and filtering (or conditioning of ECG signals) of the ECG voltages (the 12-lead ECG signals) using known noise cancelling and filtering techniques to generate a noiseless signal (or a noise free electrical graph of ECG), and thereafter calibration to standardize as per specification and digitization of the 12-lead ECG signals from its analogue form to generate the electrical graph of the 12-lead ECG (i.e. digitized ECG signals) therefrom for display and transfer thereof, wherein interpretation of the 12-lead ECG indicates the cardiovascular health of the user. Some non-limiting examples of the signal processing unit (20), includes a central processing unit (CPU), micro-processors and/or any combination of hardware or software disposed resident to or remote from the wearable ECG acquisition unit (40) and physically connected to the electrode sensor assembly (10) thereof for receiving the 12-lead ECG signals and generating the 12-lead ECG therefrom indicative of the cardiovascular health of the user.

Thus, to summarize, the wearable ECG acquisition unit (40) provided by the system (100) of the present invention facilitates non-invasive recording of the 12-lead ECG (torso ECG) that is exactly identical/equivalent to the standard 12-lead ECG (conventional ECG) but acquired from positions different than the conventional placement positions of the four limb electrode sensors (RA, LA, RL, LL). The adherence/attachment positions of the electrode sensor assembly (10) of the wearable ECG acquisition unit (40) on the user reduces movement artefacts and allows freedom of movement while performing recording of the 12-lead ECG thereby facilitating accurate ECG measurements and in turn diagnosis of critical cardiac conditions associated with the user. Further, the adherence positions of the four limb electrode sensors (RA, LA, RL, LL) on the torso region limits wiring connections within the wearable ECG acquisition unit (40) thereby imparting compactness and portability thereto with no compromise on the accuracy as compared to the conventional 12-lead ECG system, since small interconnecting wires can be deployed to place the electrode sensor assembly (10) on the torso region of the user. This portability feature allows the user to simply perform routine activities while the wearable ECG acquisition unit (40) is worn by the user and records the 12-lead ECG at the comfort of his/her home without resorting to the healthcare provider. Further, in an embodiment, the wiring is integrated within the wearable ECG acquisition unit (40) such that the connecting wires do not become tangled or pulled on by the electrode sensors, and also prevent accidental removal of the wiring from the electrode sensors. Furthermore, the adherence positions of the four limb electrode sensors (RA, LA, RL, LL) facilitates recording of the 12-lead ECG across ECG modalities (ECG types) without varying the placement (positions) of the electrode sensor assembly (10) on the user thereby eliminating need to visit the healthcare provider each time a new or a different type of ECG is to be recorded for monitoring and/or diagnostic purposes, and thus facilitates fast-tracking diagnosis and treatment of the user for the cardiac condition associated therewith. Here the term ‘ECG modality’, for the purpose of present disclosure refers to different types of ECG that are routinely recorded with objective of diagnosing different cardiac conditions of the user, and includes variations/types such as, resting 12-lead ECG (i.e. the standard 12-lead ECG), stress ECG/stress test ECG (treadmill testing), ambulatory ECG (Holter monitor) and signal-averaged ECG. In conventional ECG systems, recording of the 12-lead ECG across the ECG modalities requires the healthcare provider to manually change the placement positions of the electrode sensors on the user every time a different ECG modality is to be recorded, thereby making the whole process tedious, time-consuming and unfeasible especially when long term ECG monitoring is needed. For example, for recording of stress ECG, conventionally a Mason-Likar modification system is followed wherein the four limb electrode sensors (RA, LA, RL, LL) are transposed manually by the healthcare provider to the torso region from their conventional placement positions to ensure the user's comfort and to reduce movement artefacts while recording Another example is while recording ambulatory ECG, the no. of electrode sensors and their placement positions are changed depending upon end objective, i.e. if objective is to monitor only heart rate and rhythm then only 2-3 leads (electrode sensors) are deployed, whereas if objective is to monitor the cardiac condition, then a traditional 12-lead Holter ECG monitor is used to acquire the 12-lead ECG, wherein the 12-lead Holter ECG monitor follows the Mason-Likar modification system during recording. Another example would be during continuous bedside monitoring of ECG wherein again the Mason-Likar modification system is followed during ECG recording. It is well known to a person skilled in the art that any variations in the conventional placement positions of the electrode sensor assembly (10) on the user largely impacts accuracy and quality of the ECG signals obtained therefrom (which are of significantly lower resolution) which in turn misleads diagnosis or masks diagnosis of certain abnormalities. For example, it is known that Mason-Likar modification system during stress testing fails to determine culprit artery localization in event of myocardial infarction and ischemia. Further, due to variations in the placement positions across the ECG modalities, changes in the ECG signals recorded across one modality cannot be extrapolated to the other modality.

Here, the wearable ECG acquisition unit (40) of the present invention addresses the afore-mentioned drawbacks by proposing different placement/adherence positions of the four limb electrode sensors (RA, LA, RL, LL) which remains fixed across the ECG modalities thereby facilitating uniform acquisition of the 12-lead ECG thereacross and allowing extrapolation of ECG changes in one modality to another modality without compromising on the accuracy and quality of the ECG signals. That is, placement positions of the electrode sensor assembly (10) remain fixed/universal irrespective of the type of ECG (i.e. ECG modality) is to be recorded, thereby facilitating uniform acquisition of the ECG signals thereacross without varying said positions of the 10 electrode sensors on the user. For example, acquisition of the stress ECG using the wearable ECG acquisition unit (40) of the present invention does not require the user to manually change/alter the placement positions of any of 10 electrode sensors on the torso region of the user, which otherwise is demanded by the conventional 12-lead ECG system to reduce movement artefacts. Hence, since the electrode sensor assembly (10) is preconfigured at fixed positions on the support substrate and requires no change in adherence positions on the user, the wearable ECG acquisition unit (40) serves as a universal platform for uniform acquisition of the 12-lead ECG signals across the ECG modalities. That is, the wearable ECG acquisition unit (40) is universal with respect to the placement positions of the 10 electrode sensors irrespective of the ECG modality to be recorded and is uniform with respect to the quality (or resolution) and accuracy of the 12-lead ECG recorded which is exactly identical/equivalent to that of the standard 12-lead ECG. Thus, the universality feature of the wearable ECG acquisition unit (40) of the present disclosure facilitates recording of different types of ECG without changing the placement positions of the electrode sensors thereby making it feasible to the user to simply wear the wearable ECG acquisition unit (40) torsionally without worrying on the correctness of the electrode sensor positions affecting accuracy. Thus, the wearable ECG acquisition unit (40) serves as a standard format for acquisition of the 12-lead ECG and can thus be deployed during not limiting thereto, continuous 12-lead ECG monitoring in ICU, telemetry, and surgical procedures because it will facilitate immediate diagnosis of the cardiac conditions (for example, useful for culprit artery localization etc.) and allow prompt treatment thereof while these procedures are being performed. Usually, the conventional practise during such ongoing surgical procedures and in ICU is to keep a track of heart rhythm only rather than monitoring of the 12-lead ECG thereby missing out/delaying on diagnosis of certain cardiac conditions requiring immediate attention (like for example, heart attack) which goes unnoticed in rhythm monitoring alone. The wearable ECG acquisition unit (40) due to universality in the placement positions of the electrode sensors and recording of the 12-lead ECG equivalent in accuracy to the standard 12-lead ECG facilitates culprit artery localization in event of ischemia as against to the Mason-Likar modification system widely used for culprit artery localization with reported inability to predict precise location of coronary artery disease and high incidence of false negative in patients with ischemia. Particularly, the ECG signals are captured from the user's body via the electrode sensor assembly (10) by dynamically measuring voltage differences between two electrode sensors at particular point during heart contraction and relaxation, which are later processed and depicted graphically by the signal processing unit (20) in the form of the 12-lead ECG representative of the cardiovascular health of the user. In addition, due to sharing of the location co-ordinates of the user, the wearable ECG acquisition unit (40) also helps in mobility tracking of the user and allows the user to seek immediate medical attention from the healthcare provider located remote to the user based upon the location information thereof. Hence, the wearable ECG acquisition unit (40) facilitates diagnosis of heart attack and other related cardiac abnormalities with great accuracy and speed. Due to a comfortable wear and being suitable to wear all day or while sleeping, the wearable ECG acquisition unit (40) can be used for long term ECG monitoring. The wearable ECG acquisition unit (40) provided by the system (100) of the present disclosure thus functions as a standalone unit and is made easily available in local market for purchase by the user/patient to self-monitor the health condition from the comfort zone of home.

It is to be noted herein that although the wearable ECG acquisition unit (40) with the electrode sensor assembly (10) principally having only the ECG sensors are envisioned herein, the wearable ECG acquisition unit (40) may be configured to have a plurality of other types of sensors (for monitoring of other physiological conditions such as, for example, glucose levels, blood pressure, body temperature etc.) including, but not limited thereto, non-electrical sensors, physiological type sensor (such as motion sensor, temperature sensor etc.) and like or combinations thereof.

Forms, Materials and Sizes

The wearable ECG acquisition unit (40) is implemented/fabricated in at least one form (or shape) selected from a group consisting of, but not limited thereto, a vest, a jacket, a pad, a garment, a pador band, a shirt, a strip, a strap, a band, a belt, a plate, a patch, a mask, a harness and like, and combinations thereof. In one embodiment, the wearable ECG acquisition unit (40) is fabricated in a single, or unitary form (for example, a fabric garment such as shirt etc.) wherein, the electrode sensor assembly (10) along with the electrical connections and the signal processing unit (20), and all other components associated therewith are fabricated into a one single wearable unit. In another embodiment, the wearable ECG acquisition unit (40) is fabricated as an ensemble of the afore-stated forms, (for example, a jacket with one/more belts etc. adapted to be worn together) and may include some portion being removable (or disposable) such as, for example, having removable sensors that would facilitate use of other physiological sensors. In an embodiment, the wearable ECG acquisition unit (40) is fabricated to be reusable (suggesting multiple uses) or disposable (suggesting a single use).

The support substrate which forms the body of the wearable ECG acquisition unit (40) is made up of any appropriate non-electrical conducting material known in the art. In one embodiment, the support substrate is formed using a flexible (or stretchable/expandable) and elastic/elastomeric material known in the art wherein, the flexibility feature offers comfort and provides optimal fit to the user to account for different torso sizes thereof by providing a one-size-fits-all product imparting a comfortable wear. In another embodiment, the support substrate is formed using a non-stretchable, rigid or a semi-rigid material known in the art. Preferably, the support substrate is made up of, not limiting to, any strong and lightweight synthetic or foam material or any other material type known in the art, and may include multiple layers (or thickness) of such materials having different characteristics/purposes thereby making a comfortable wear to the user during long term monitoring.

The wearable ECG acquisition unit (40) is sized and shaped, and is ergonomically adaptive to fit anatomy and gender of the user of different sizes such that it conforms about the torso region thereof when worn and allows adherence of the electrode sensor assembly (10) on the shoulder, the chest and the abdominal region for acquisition of the 12-lead ECG signals therefrom. In one embodiment, the wearable ECG acquisition unit (40) is constructed using stretchable material to accommodate different grades of human torso sizes. In another embodiment, the wearable ECG acquisition unit (40) is constructed in customized sizes and dimensions to cater to the requirements of the user (men, women, children etc.) and provide a comfortable wear thereto. In yet another embodiment, the wearable ECG acquisition unit (40) constructed with electrical wired sensors (i.e. electrode sensor provided with extendable wires to facilitate their manual attachment on the user's body), facilitates size adjustment whilst permits the user to freely roam without encumbrance of wires. Particularly, the wearable ECG acquisition unit (40) is designed such that it provides adequate contact of the electrode sensor assembly (10) to the skin of the wearer even during exercise thus allowing the electrode sensor assembly (10) to acquire adequate ECG signal quality.

The electrode sensor assembly (10) of the present disclosure is made using any appropriate electrical conducting material known in the art to facilitate sensing of the 12-lead ECG signals from the user. In an embodiment, sensing portion of the electrode sensors is formed using any known comfortable material that requires little or no skin preparation or adhesive unlike the conventional 12-lead ECG system and the material that offers high resistance to sliding movement of the electrode sensors against the skin of the wearer to ensure stable and continuous acquisition of the 12-lead ECG signals therefrom. In another embodiment, the 10 electrode sensors (or ECG sensors) of the present disclosure are manufactured in any shape (for example, triangle, circle, square, strip etc.), size (or dimension) and using any electrical conducting material known in the art to facilitate detection of the ECG signals in a non-invasive manner-either by the direct contact/attachment/adherence to the skin surface of the user (for example, by way of integration within the wearable system, or using adhesives/fasteners to directly affix the electrodes on the skin surface of the user) or by the indirect contact/adherence.

The ECG sensors of the present disclosure are of type known in the art and selected from, but not limiting thereto, wet electrode sensors to dry sensors, textile-based sensors, knitted integrated sensors (KIS), planar fashionable circuit boards, gel-based sensors, foam-based sensors, conductive polymer-based sensors, metallic/metal plate-based sensors and like or combinations thereof. In one embodiment, the ECG sensors of the present disclosure are based on Ag/AgCl or polydimethylsiloxane. In another embodiment, preferably dry electrode sensors coated with conductive gel are used to acquire strong and quality ECG signals.

However, it is evident to a person skilled in the art that the wearable ECG acquisition unit (40) can be fabricated in any form, shape, size, dimension and using any of the support substrates already known in the art that enables a comfortable wear to the user, thus contributing to a long-term wear and versatility.

Following are few non-limiting example embodiments of the wearable ECG acquisition unit (40) fabricated in varied forms in accordance with the present disclosure.

### Example Embodiment 1

The wearable ECG acquisition unit (40) is implemented as a vest having a patch affixed thereon. The vest is configured to be worn like a jacket around the torso region of the user subsequent to which the 10 electrode sensors preconfigured on the vest disposes and adheres to their respective positions on the chest and the abdominal region of the user to acquire the 12-lead ECG signals therefrom and generate the 12-lead ECG.

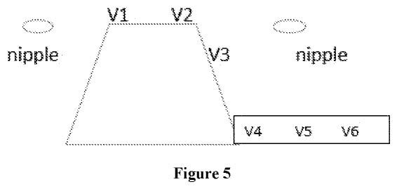

The vest is constructed using the support substrate made up of a flexible/stretchable fabric to account for different torso/body sizes (one-size-fits-all). Alternatively, the vest constructed using the stretchable fabric is made available in few standard sizes such as—small, medium, and large. The four limb electrode sensors (RA, LA, RL, LL) are integrated at fixed predetermined positions within the stretchable fabric such that they dispose at their said positions (in accordance with the present invention) on the shoulder (near collar bone) and the abdominal region (near umbilicus) of the user subsequent to wearing of the vest. Alternatively, the user manually attaches the four limb electrode sensors (RA, LA, RL, LL) at said positions since these positions are easily identifiable by a lay person. A rhomboidal-shaped patch with the six precordial electrode sensors (V1-V6) is separately constructed using appropriate material as shown in FIG. 5, wherein, upper side (or portion/surface) of the patch is configured with the electrode sensors V1 and V2, lower side/surface is configured with the electrode sensors V4, and V5, V6 separately affixed as a metal plate horizontal extension, whereas the electrode sensor V3 is configured across the surface connecting the upper and lower sides/surfaces. V1 and V2 are disposed for being adhered onto the user at fourth intercostal space on either side of the sternum, and the fourth intercostal space is where the nipples of the user usually lie, and hence the nipples serves as a guideline for placement/alignment of said patch onto the user so that once V1 and V2 aligns properly, rest of the precordial electrode sensors automatically gets aligned at their corresponding positions on the user. That is, the said patch is so constructed such that when the upper portion of the patch is aligned alongside the nipples at centre thereof (which serves as a guideline for placement of the patch), the six precordial electrode sensors (V1-V6) disposes at said positions on the chest region such that simple pressing of the patch facilitates acquisition of the precordial ECG signals therefrom. Alternatively, the patch is constructed to have 2 extendable (or expandable) strips—one having the electrode sensor V3 and other having the electrode sensors V4-V6. In such case, after placement of the patch in-line with the nipples at the centre thereof, the user has to expand/collapse these extendable strips allowing placement of the six precordial electrode sensors (V1-V6) integrated therewithin at said positions on the chest region of the user. The patch after fabrication is affixed onto the vest on surface facing the skin of the user such that when the vest is worn by the user, the four limb electrode sensors (RA, LA, RL, LL) and the six precordial electrode sensors (V1-V6) disposes at said positions on the user to acquire the 12-lead ECG therefrom when pressed against the skin thereby obviating need of identifying positions of the electrode sensors near the shoulder and the umbilicus region by the user. The user is required to wear the vest like a jacket and fasten it with belts to apply pressure sufficient enough to establish close contact of the electrode sensors with the skin of the user at said positions thereon for acquisition of the 12-lead ECG signals therefrom. The vest being light in weight is inconspicuous under most clothes and thus can be used regularly while performing routine activities. Alternatively, the vest is fabricated using the support substrate made up of a rigid material in the form of a pad, wherein placement of the pad on the torso region of the user and thereafter application of an external force facilitates acquisition of the 12-lead ECG.

### Example Embodiment 2

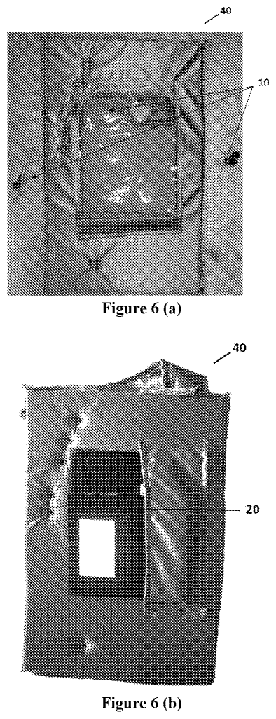

The wearable ECG acquisition unit (40) is implemented in the form of a belt as shown in FIG. 6 for applying to the user's body and for obtaining and transferring the 12-lead ECG signals to the signal processing unit (20) for generation and transmission of the 12-lead ECG therefrom. The belt is constructed using the support substrate of semi-rigid material and preconfigured with the electrode sensor assembly (10) electrically coupled to the signal processing unit (20) disposed resident thereto. The belt is constructed such that when worn around the torso region of the user, the six precordial electrode sensors (V1-V6) automatically gets placed at their positions on the chest region, whereas the four limb electrode sensors (RA, LA, RL, LL) are required to be manually attached by the wearer to their positions using extendable wires, as can be seen from FIG. 6 (a), to facilitate recording of the 12-lead ECG of the user. Additionally, as can be seen from FIG. 6 (b), a Velcro arrangement is provided to closely secure the belt around the torso region of the wearer such that the electrode sensor assembly (10) gets placed at said positions thereon to acquire the 12-lead ECG signals therefrom. Particularly, the Velcro arrangement helps in reducing motion artefacts associated with slight movement between the electrode sensor assembly (10) and the skin of the wearer thereby allowing acquisition of accurate and quality ECG signals therefrom.

### Example Embodiment 3

The wearable ECG acquisition unit (40) is implemented as a unitary unit in form of a garment constructed using flexible material as the support substrate. The electrode sensor assembly (10) is preconfigured at positions fixed within the garment such that when the garment is worn by the user, the 10 electrode sensors gets placed on the chest and the abdominal region at their respective positions on the skin to establish concomitant contact therewith to acquire the 12-lead ECG signals therefrom for generating the 12-lead ECG of the user.

The plurality of communication device (80), hereinafter referred to as “the communication device (80)”, is operatively coupled to the wearable ECG acquisition unit (40) via the second component to receive the 12-lead ECG therefrom. Specifically, the communication device (80) is operatively coupled to the signal processing unit (20) for receiving the 12-lead ECG wirelessly transmitted therefrom. The communication device (80) essentially is a computing device and comprises of components, not limiting thereto, at least one processor, at least one memory, an audio-visual unit, and at least one input-output unit and wired or wireless network capabilities, all of the components operably coupled to each other, wherein the afore-stated components implement various functionalities known in the art. In an embodiment, the processor facilitates execution of processor-executable instructions stored in the memory, the memory includes any computer-readable storage media and stores computer instructions such as the processor-executable instructions for implementing the various functionalities of the system (100) as well as any data relating thereto, generated thereby, or received via any communication interface or input device, the audio-visual and the output unit allows information to be visualized/viewed or otherwise perceived in connection with execution of the instructions, whereas the input unit allows the user to perform manual selections, enter data or any other information, or interact in numerous ways with the processor during execution of the instructions. The processor of the communication device (80) is further coupled to the communication module (or the transmitting module) to transmit and/or receive information to and from an external device/unit over a communication network (60), preferably a wireless communication network (60). Some non-limiting examples of the communication device (80) includes smartphone, tablet computer, laptop computer, personal computer, desktop, personal digital assistant (PDA) or any other handheld or worn computing device. The communication device (80) is further configured with a web application to facilitate real-time interaction and remote exchange of information between the external device/unit and the communication device (80) and therewithin. Specifically, the signal processing unit (20) of the wearable ECG acquisition unit (40) remotely communicates over the communication network (60) with the communication device (80) via the web application executing thereon to facilitate wireless remote transmission of the 12-lead ECG thereto. In an embodiment, the system (100) includes a plurality of healthcare providers (for example, a doctor) each being associated with the communication device (80) (i.e. healthcare provider communication device) and a plurality of users (for example, a patient) each being associated with the communication device (80) (i.e. user communication device), wherein the web application configured on the communication device (80) displays/presents information collected by the wearable ECG acquisition unit (40) and establishes audio-visual communication between the user communication device over the communication network (60) with the communication device (80) of any of the plurality of healthcare providers to receive opinion/medication (prescription) and/or treatment therefrom thereby enabling wireless remote monitoring of the cardiovascular health of the user by the healthcare provider in real-time. The web application configured on the user communication device provides a user interface whereas that configured on the healthcare provider communication device provides a healthcare provider interface for displaying analysis results and ECG data of the user to provide opinion/medication thereto by the healthcare provider. Particularly, the user interface and the healthcare provider interface are two different interfaces of the web application which essentially is the same. The user interface provides access to view only the ECG data and/or other medical information of the wearer along with details of the plurality of healthcare providers registered onto the web application i.e. user can access only their medical data and the healthcare provider's information, whereas the healthcare provider interface provides access to view data recorded and stored from the plurality of users/wearers i.e. displays vital data from multiple users or wearers. In an embodiment, each user is associated with a unique identifier (ID) such that data from multiple user's is stored against the unique identifier within a centralized database of the web application for reference and viewing by the healthcare provider. In a preferred embodiment, the wearable ECG acquisition unit (40), and specifically the signal processing unit (20) thereof is communicatively coupled over the communication network (60) to the user/wearer communication device (80) via the web application configured thereon to facilitate wireless transmission of the 12-lead ECG thereto. In an embodiment, the system (100) provides for self-registration of the wearable ECG acquisition unit (40) by the user/wearer. Here, self-registration is a process by which the wearable ECG acquisition unit (40) is associated/linked (or paired) over the communication network (60) with the communication device (80) of the wearer or user (for example, the patient) of the system (100). Additionally, the user is required to self-register himself on the web application using the user communication device by creating the unique ID and providing information such as, not limiting to, personal details/general information and cardiac/medical history along with storing a baseline ECG (template 12-lead ECG) recorded using the wearable ECG acquisition unit (40) linked to the unique ID of the user. The baseline ECG serves as a template for future comparisons to detect/note any changes/abnormalities in the ECG readings when the user shows any abnormal symptom's. The self-registration process associates the wearable ECG acquisition unit (40) with the user, wherein each user is associated with the unique identifier, and the data is stored against that identifier within the centralized database of the web application for future reference, visualization and comparison by the healthcare provider. In an embodiment, any authorized third party can access the wearer's user interface hosted on the web application by feeding the user account details (or login credentials/unique identifier) through the communication network (60) (for example, internet) from a remote or any other external computing device for viewing the medical and/or other information associated with the user or wearer. In addition, the healthcare provider is also required to self-register (i.e. account creation using login credentials) himself/herself on the web application using the healthcare provider communication device by providing information such as, not limiting to, personal details/general information and area of expertise/qualifications along with their location details which are stored within the centralized database of the web application. Thus, the centralized database configured on the web application includes complete details of the plurality of users and the healthcare providers who have undergone the self-registration process. This allows the user to manually select the healthcare provider as per the user's preference to whom the medical information/data should be transferred/forwarded/shared remotely over the communication network (60) for interpretation (or evaluation) and advice/guidance on the ECG data. In an embodiment, the web application is configured to facilitate automatic routing of the user to the nearest located healthcare provider automatically selected from the centralized database and based on the location co-ordinates information of the user, in case of an emergency thereby allowing the healthcare provider to carry on a two-way communication with the user or wearer and immediately address the concern and offer appropriate remedy/treatment regimen. In such an event, the concerned remotely located and available healthcare provider will be notified upon receipt of the ECG data of the user on the healthcare provider communication device over the communication network (60). In an event the healthcare provider does not respond back within a pre-defined/pre-set time limit, the ECG data of the user will be transmitted to another available and remotely located healthcare provider for immediate action and remedy. The healthcare provider can remotely view the ECG data of the user along with any historical data associated therewith and stored within the centralized database to perform comparison and prognosis through the healthcare provider interface on the communication device (80) coupled thereto and thereafter input-in comments and advice or choose from pre-loaded pop-up menu to provide expert comments and opinion over the communication network (60) to the user on the communication device (80) coupled thereto thereby facilitating wireless remote monitoring of the cardiac status of the user by the healthcare provider located remote. In an embodiment, the 12-lead ECG and components thereof, such as the P wave, T wave, and QRS complex and their amplitudes, widths etc. are extracted, compared, analyzed and stored as ECG features within the centralized database to facilitate the healthcare provider to extract and analyze to identify the cardiac markers associated therewith. Particularly, by allowing comparison of real-time ECG readings to the historical data for the same user present within the centralized database of the web application, an accurate prognosis can be made by the healthcare provider and evidence-based symptom management can be recommended for timely interventions in a tailored manner which is specifically beneficial during long-term monitoring of the user. In an alternate embodiment, however, a centralized web server may be separately provided with the web application executing thereon such that the signal processing unit (20) transmits all information to the centralized web server, and the web application executing thereon routes the data to the healthcare provider located remote. The user can then access medication prescribed by the healthcare provider by login into the user interface of the web application hosted on the centralized web server using the user communication device over the communication network (60).

The communication module is operatively coupled to the components of the system (100) for establishing the communication network (60) to facilitate flow of information therebetween thereby allowing real-time monitoring and management of the cardiovascular health of the user. The communication module allows the components connected over the communication network (60) to share content, information and enables interactive communication therebetween. The communication module is coupled to the signal processing unit (20) ( ) and the communication device (80) facilitating exchange of information/data and interaction over the communication network (60) therebetween. In an embodiment, the communication module is anyone of a wired and a wireless communication network module establishing a wired and a wireless communication network respectively. In a preferred embodiment, the wireless communication network module is deployed for establishing the wireless communication network within the components of the system (100). In another embodiment, the wireless communication network includes but not limiting thereto, a GSM (Global System for Mobile Communications)—GPRS (General Packet Radio Service), infrared, satellite communication, Wi-Fi, RF (Radio Frequency), local area network, wide area network, Bluetooth network, aggregated networks such as the internet and cloud-based computer network or any similar wireless communication technology and combinations thereof. In a non-limiting example embodiment, a Bluetooth module is configured on the signal processing unit (20) and the user communication device to facilitate wireless transmission of the 12-lead ECG therebetween. Specifically, the Bluetooth module configured on the signal processing unit (20) is continuously synced with the Bluetooth module configured on the user communication device for real-time transmission of the 12-lead ECG signals therebetween. In another embodiment, the healthcare provider communication device is communicatively coupled to the user communication device via the web application over a WiFi communication network thereby facilitating flow of information therebetween thus allowing wireless remote monitoring of the user. Thus, the communication network (60) allows transmission of the user's ECG data to the web application configured on the user communication device and in turn to the healthcare provider communication device facilitating remote viewing of the ECG data by the healthcare provider to receive interpretation and opinion therefrom thereby enabling effective wireless monitoring and management of the cardiovascular health of the user by the healthcare provider located remotely.

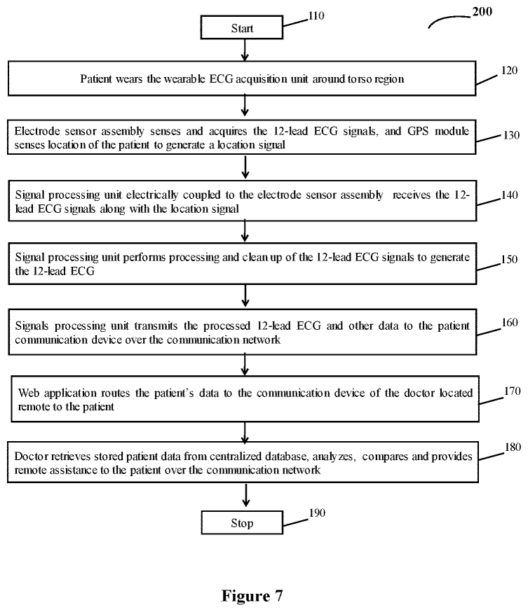

Referring to FIG. 7, in another aspect, the present disclosure provides a method (200), (hereinafter referred to as “the method (200)”) for 12-lead ECG recording and wireless remote monitoring. The method (200) facilitates recording of the 12-lead ECG equivalent to the standard 12-lead ECG and thereafter wireless transmission of the 12-lead ECG to the healthcare provider located remotely thereby facilitating real-time wireless remote monitoring and management of the cardiovascular health of the user by the healthcare provider. The method (200) operates and is explained in conjunction with the components/parts of the system (100).

The method (200) starts at step (110). At step (120), the method (200) includes wearing of the wearable ECG acquisition unit (40) by the user or patient around torso region thereof subsequent to which the electrode sensor assembly (10) gets torsionally placed/positioned at their respective locations/points on the user i.e. particularly the six precordial electrode sensors (V1-V6) gets automatically placed and adhered to their conventionally advocated placement positions on the chest (or precordial) region to acquire the precordial ECG signals therefrom, whereas the four limb electrode sensors (RA, LA, RL, LL) gets automatically placed and adhered at their proposed positions as per the present invention on the chest (specifically near shoulder i.e. collar bone region) and the abdominal region (near umbilicus) of the user to acquire the ECG signals therefrom, thereby collectively being disposed to facilitate acquisition of the 12-lead ECG signals from the user. Specifically, the four limb electrode sensors (RA, LA, RL, LL) gets automatically placed and adhered such that RA and LA gets disposed to lie within or adjacent to right and left delto-pectoral groove respectively below lateral end of clavicle, whereas RL and LL gets disposed to lie at a distance ranging from 1-3 inches above and left lateral to the umbilicus of the user at 12 o'clock and 3 o'clock position respectively to acquire the ECG signals therefrom upon wearing of the wearable ECG acquisition unit (40) by the user. However, in an alternate embodiment, the method (200) includes manually placing the four limb electrode sensors (RA, LA, RL, LL) by the wearer at their proposed aforementioned positions being easily identifiable on the shoulder and the abdominal region pursuant to wearing of the wearable ECG acquisition unit (40) by the user.

At step (130) the method (200) involves sensing and acquisition of the 12-lead ECG signals by the electrode sensor assembly (10) positioned on the torso region of the user. In an embodiment, acquisition of the 12-lead ECG signals by the electrode sensor assembly (10) is in real-time and can be continuous (thus allows for continuous monitoring of the user), or at regular intervals (facilitating intermittent/periodic monitoring) such as hourly, daily, weekly and any interval in between, or can be on demand, both of which may or may not be coincided with symptomatic complaints (ex. Breathlessness etc.) from the users. For example, a button may additionally be provided on the wearable ECG acquisition unit (40) such that manual operation/tapping (or pressing) of the button by the wearer (for example, in event of any cardiac trouble, or upon facing any adverse event) facilitates acquisition/recording of the 12-lead ECG signals on demand. In addition, the GPS module also senses location of the user to generate a location signals.

At step (140) the method (200) involves transmission of the 12-lead ECG signals (along with the location signals) by the electrode sensor assembly (10) to the signal processing unit (20) electrically coupled thereto. In an embodiment, data transmission between the electrode sensor assembly (10) and the signal processing unit (20) is preferably a wired transmission via electrical connections there between, or alternatively is a wireless data transmission. In another embodiment, transmission can be real-time and continuous, or at regular intervals or on-demand.

At step (150) the method (200) includes processing and clean-up of the 12-lead ECG signals by the signal processing unit (20) to generate the 12-lead ECG therefrom which is indicative of the cardiovascular health of the wearer.

At step (160) the method (200) includes transmission of the generated 12-lead ECG (along with the user's location signals) by the signal processing unit (20) to the user/patient communication device (80) operatively coupled thereto over the communication network (60). Preferably, data transmission between the signal processing unit (20) and the user communication device, happens remotely over the wireless communication network (60). The 12-lead ECG (and data related thereto) along with the location signals (or co-ordinates) are stored in real-time against the unique identifier (ID) of the user within the centralized database of the web application configured on the user communication device. Thereafter, the data is displayed and can be viewed on the user communication device using the user interface of the web application configured therein.

At step (170) the method (200) includes automatic routing of the user data by the web application to the healthcare provider (doctor) communication device (80) located remote to the user based upon the location co-ordinates shared over the communication network (60). In an alternate embodiment, the user/wearer manually selects the healthcare provider from the shared list stored within the centralized database to whom the user's data should be transferred remotely to receive the medication. In an embodiment, the healthcare provider is notified (for example, via pop-ups, alarms, alerts, buzzing etc.) upon receipt of the user's data (including the 12-lead ECG and other data related thereto) on the healthcare provider communication device, and is provided with a pre-set time period to respond back to the user, after which the user's data gets automatically transferred to another healthcare provider located remote to the user.

At step (180) the method (200) includes retrieving of the user's/patient's data (and other historical data associated and therewith) by the healthcare provider using the healthcare provider communication device from the centralized database of the web application configured therein. The healthcare provider remotely accesses and visualizes the user's data over the communication network (60) using the healthcare provider interface of the web application executing on the healthcare provider communication device, remotely interprets the user's data and thereafter provides remote medical assistance and medication on the user communication device coupled thereto over the communication network (60) for real-time monitoring and management of the cardiovascular condition/health of the user. In an embodiment, the healthcare provider is provided with incentives/monetary benefits in exchange for the medical assistance provided to the user. The method (200) ends at step (190).

Hence, the method (200) facilitates wireless remote monitoring i.e. wireless transfer to remote interpretation and management of the cardiovascular health of the user by the healthcare provider over the communication network (60) thereby eliminating the requirement of paying physical/in-person visits to any healthcare facility and/or the healthcare provider by the user. The present invention thus facilitates in providing immediate medicament to the users residing in rural areas, wherein each doctor may be provided with the wearable ECG acquisition unit (40), who when visited by the user can record and transmit ECG to a cardiologist/specialist registered on the web application to receive opinion therefrom and share it with the user. This will thus avoid the user to pay a visit to the specialist suggested by family doctor, thereby saving time from symptom onset to treatment.

Various studies were performed to validate the claim that the 12-lead ECG recorded from the proposed torso positions of the 10 electrode sensors according to the present invention is comparable and is equivalent to the standard 12-lead ECG obtained from the conventionally advocated placement positions of the 10 electrode sensors.

Example Sample Case Study

Aim and Objective:

To study the utility of torso-ECG (t-ECG) versus conventional ECG (c-ECG) for accurate diagnosis of Acute Coronary Syndrome (ACS), and to ascertain whether the t-ECG miss or over-diagnose ACS changes.

The following study was carried out to validate the 12-lead ECG recorded from the torso positions of the 10 electrode sensors against the standard 12-lead ECG with special focus on Acute Coronary Syndrome (ACS).

Here, the term ‘torso-ECG (t-ECG)’ refers to acquisition of the 12-lead ECG by the 10 electrode sensors positioned on torso only region (i.e. trunk region) of the user as proposed by the present invention, wherein, the six precordial electrode sensors (V1-V6) are positioned on the conventionally advocated placement positions thereof whereas the four limb electrode sensors (RA, LA, RL, LL) are positioned on the torso only region according to the present invention (as opposed to their conventional placement positions), i.e. upper limb electrode sensors (RA, LA) are placed in the respective delto-pectoral grooves below the lateral end of the clavicle, and right lower limb electrode sensor (RL) is placed 2 finger breadths above the umbilicus at 12 o'clock positions while left lower limb electrode sensor (LL) is placed 2 finger breadths left lateral to the umbilicus at 3 o'clock position with respect to the umbilicus of the user. The term ‘conventional ECG (c-ECG)’ refers to acquisition of the 12-lead ECG by the 10 electrode sensors positioned on the conventionally advocated placement positions thereof on the user.

Design and Intervention:

The 12-lead ECGs were recorded by both techniques (c-ECG & t-ECG) in 1361 patients from the coronary care unit & out-patient department of a tertiary care hospital. The 12-lead ECGs were recorded by trained technicians or nurses on GE Mac 1200 machine. The c-ECG was recorded by placing the 10 electrode sensors at the conventionally advocated placement positions thereof on the user. The t-ECG was recorded by placing the four limb electrode sensors (RA, LA, RL, LL) specifically at torso only positions proposed by the present invention (FIGS. 3 and 4). Positions of the six precordial electrode sensors (V1-V6) were the conventional placement positions thereof. The ECG was recorded with breath-holding for a few seconds to avoid beat to beat changes in the QRS amplitude that occur due to respiratory abdominal movements. Patients with ACS whose ECGs had dynamic changes while in hospital had multiple ECGs recorded. A total of 1526 sets of ECGs (each set consisting of one c-ECG and one t-ECG) were analyzed. Two trained cardiologists blinded to the ECG recording method made the ECG analysis/diagnoses. The ECGs (c-ECG & t-ECG) were provided to them in random fashion. All the ECG waveform measurements were made manually, using a hand-held magnifying lens to the nearest 0.25 mm both vertically and horizontally. Standard ECG criteria were used for the diagnosis of ACS. There were 457 ECG sets from 342 patients with ACS. Patients with anterior wall myocardial injury whose ECGs did not show changes in the limb leads were excluded. Hence, 116 ECG sets from 112 patients of anterior infarction who had ST segment changes restricted to precordial leads were excluded. Finally, 341 ECG sets from 230 patients with ACS and 324 sets of patients diagnosed to be normal on c-ECG were considered for the purpose of this study.

Statistical Analysis

The c-ECG was considered as the ‘gold standard’ against which the t-ECG was compared. The comparison between c-ECG and t-ECG diagnosis was quantified in terms of the number of ECGs that were a diagnostic match between t-ECG and c-ECG. Based on the number of diagnostic matches and differences, we intend to calculate Clopper-Pearson binomial confidence intervals for sensitivity and specificity, based on the exact correct distribution rather than an approximation standard logit confidence intervals given by Mercaldo et al, if required.

Results

From the 230 ACS patients, 341 sets of ECG were studied for ECG changes in inferior or lateral myocardial areas. Sub-categorization of ACS was done as per leads showing ST elevation or depression. As a control, 324 ECG sets were also analyzed, where the c-ECG had been diagnosed as normal (Table 1). Thus, a total of 665 ECG sets were used for subsequent analysis.

TABLE 1
Comparison of ACS ECGs with normal ECGs.

c-ECG

| | ACS (341) | | | Normal (324) | | |
|---|---|---|---|---|---|---|
| | Yes | No | Total | Yes | No | Total |
| t-ECG | 341 | 0 | 341 | 324 | 0 | 324 |

Of the ACS ECGs, ST elevation was seen in 234 ECGs and ST depressions 154 ECGs. All 341 ECG sets from the 230 patients with ACS changes on c-ECG were diagnosed correctly by t-ECG as well, achieving 100% sensitivity (Clopper Pearson CI: 98.89-100%). Similarly, all the 324 ECGs diagnosed as normal on c-ECG, were correctly labeled/identified as normal by t-ECG, thereby achieving 100% specificity (Clopper Pearson CI: 98.87-100%). The localizations of ST elevation and ST depression were also accurately diagnosed by the t-ECG. The distributions of ST elevation and ST depression were identical in c-ECG and t-ECG (Table 2).

TABLE 2
Territory wise distribution of ECG changes in ACS.

| | | c-ECG | | | | | |
|---|---|---|---|---|---|---|---|
| | | ST elevation | | | ST depression | | |
| | | Inferior | Lateral | Inferolateral | Inferior | Lateral | Inferolateral |
| t-ECG | Yes | 189 | 43 | 02 | 107 | 43 | 04 |
| | No | 0 | 0 | 0 | 0 | 0 | 0 |

Note:
When there was ST elevation and ST depression in the same ECG, it was included in the both categories.

In 189 cases, the c-ECG was diagnosed as inferior wall STEMI; in 43 patients, lateral wall STEMI; in 2 patients, inferolateral wall STEMI (ST-segment elevation myocardial infarction). These c-ECG interpretations were identical with the t-ECG diagnoses, thereby validating that t-ECG is comparable to c-ECG.

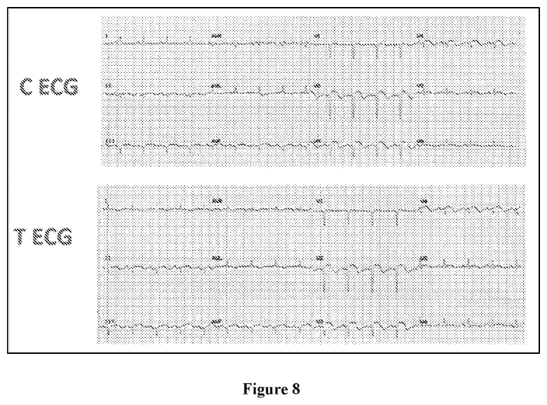

Representative examples comparing t-ECG with c-ECG in different subsets of ACS are shown in FIGS. 8-10.

FIG. 8 relates to an evolved anterior wall myocardial infarction due to non-proximal occlusion of the left anterior descending artery (as evidenced by ST coving in the inferior leads). Note: The R wave amplitude in lead I is higher in c-ECG while the S amplitude in lead II is higher in the t-ECG; however, the diagnosis remains unchanged.

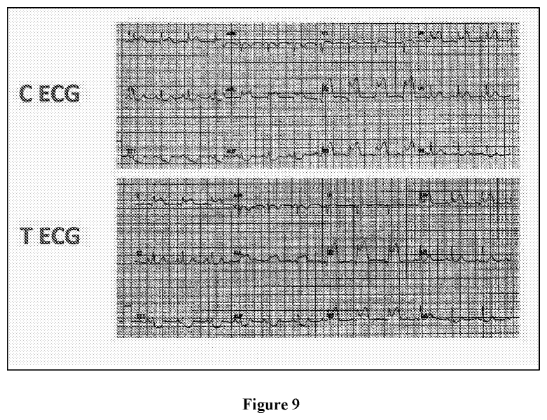

FIG. 9 relates to an acute anterior wall myocardial infarction due to proximal occlusion of the left anterior descending artery, as evidenced by with ST depression in the inferior leads. Note: The QRS complex amplitude is more in the inferior leads in the t-ECG, though the diagnosis remains unchanged.

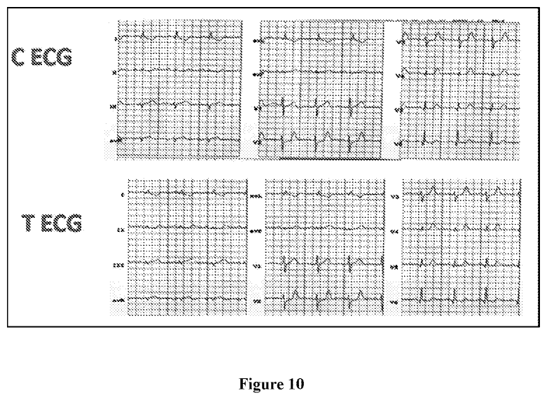

FIG. 10 relates to an acute evolving inferior wall myocardial infarction. Note that even the subtle early ST elevations are picked up by the t-ECG.

The following were some of distinctive features on c-ECG which were reproduced on t-ECG.
1. ST elevation in anterior and inferior leads (FIG. 8).
2. ST depression in inferior lead changes in anterior wall MI (myocardial infarction) (FIG. 9).
3. Subtle ST elevations in acute evolving inferior wall STEMI (FIG. 10).

In some of the ECGs acquired, as shown in FIGS. 8 and 9, there were minor differences in the amplitude of R or S wave differed between c-ECG and t-ECG; however, the ECG diagnosis remained unchanged. It is also obvious from ECG figures that ECG morphology in precordial leads is identical in both c-ECG and t-ECG.

Conclusion

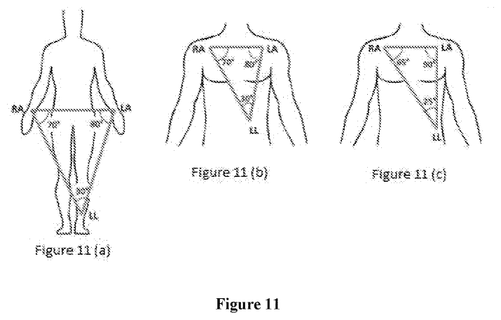

The study conclusively establishes that the t-ECG is accurate as the c-ECG with respect to diagnosis of ACS. Although R and S wave amplitude differed between c-ECG and t-ECG as can be seen from the graphical representations of ECGs in FIGS. 8-10, it did not have any impact on final ECG diagnosis of particular type of ACS. The present study had included a large number of patients with ST depression and ST elevation in inferior or lateral leads, and importantly none of these were missed on t-ECG. Similarly, in 324 normal c-ECGs there was not a single false positive finding for ACS in the t-ECG. This absence of false positivity and false negativity in using the proposed torso only positions of the present invention were attributed to the fact that the three angles of the Einthoven triangle in the proposed torso only positions are similar to that of the conventional placement positions, as can be seen in FIG. 11. Hence, it was found that the 12-lead ECGs acquired from the proposed torso only positions of the four limb electrode sensors (RA, LA, RL, LL) is comparable to the standard 12-lead ECGs (c-ECG) with respect to 100% accuracy in identification and characterization of ACS. Thus, the invention significantly reduces symptom to door time in STEMI and helps in screening out the non-significant ECG changes thereby saving time of patients and physicians alike.

Therefore, the system (100) and the method (200) of the present disclosure facilitates recording of the 12-lead ECG equivalent to the standard 12-lead ECG and further allows wireless remote monitoring i.e. facilitates wireless transfer of ECG data of the user to remotely interpret and manage the cardiovascular health thereof by the healthcare provider located remote to the user. Although slight variations in the ECG waveforms in the 12-lead ECG (torso ECG) when compared with the standard 12-lead ECG are foreseen, the system (100) and the method (200) of the present disclosure offers accuracy equivalent to that of the conventional 12-lead ECG system with respect to diagnosis of the cardiovascular disorders i.e. the 12-lead ECG (torso ECG) does not miss on diagnosis of the cardiovascular disorders/events which are otherwise detected/noticed by interpreting the standard 12-lead ECG. The system (100) and the method (200) performs non-intrusive yet accurate recording of the 12-lead ECG and simple monitoring in real-time for extended time period to track the cardiac activity of the user remotely. The system (100) and the method (200) is wireless, un-obstructive and provides real-time wireless and remote monitoring of physiological functions that seamlessly integrates in a daily life of the user over longer periods of time, thereby facilitating intensive management of on ongoing disorder (or diseased state) through pharmacological means or preventive interventions of a developing disease (that may or may not be symptomatic to the user). The wearable component provided by the system (100), i.e. the wearable ECG acquisition unit (40) incorporates features that significantly improve wearability, performance and patient comfort throughout an extended monitoring period. The wearable ECG acquisition unit (40) is small, light-weight in construction and thus can easily be worn by the wearer during period of exercise and data can be collected and analyzed while the wearer participates in their normal or day-to-day activities or even while the patient is asleep. The wearable ECG acquisition unit (40) non-invasively evaluates the wearer's health condition in a matter of seconds without affecting the wearer such that certain conditions can be controlled, or predicted, before they actually occur. The wearable ECG acquisition unit (40) of the system (100) of the present disclosure which works as the standalone unit is designed to be universal, smart, light weight, mobile and cable free thereby making it portable and wearable. Further, the wearable ECG acquisition unit (40) is designed to be inexpensive and is made readily available for early identification of, not limiting to, cardiac and pulmonary disease at comfort of the user's home without resorting to specialized medical care facility or skilled physician thereby eliminating the need for the user to pay frequent visits to the healthcare facility (for ex. hospital) every time a new ECG is to be recorded. The wearable ECG acquisition unit (40) carries out a variety of ECG based cardiac procedures such as, for example, cardiac rhythm monitoring, event monitoring, 12-lead monitoring etc.

The wearable ECG acquisition unit (40) provided by the system (100) of the present disclosure is made easily available in local market for purchase by the user/patient to self-monitor the health condition from the comfort zone of home. The system (100) and the method (200) can be used universally for performing recording of ECG in both ambulatory and home-based settings without making any change in the placement positions of the electrode sensors, and perform wireless remote monitoring of the user across any location by the healthcare provider while the user continues with his/her daily activities without any interference. The system (100) and the method (200) reduces the time from symptom onset to arrival at the healthcare facility, and door to reperfusion time in treatment of the cardiovascular condition is also reduced significantly since the user is monitored and managed by the healthcare provider remotely, since the 12-lead ECGs can be sent to any location over the communication network (60) and expert opinion can be sought almost immediately. The time from symptom onset to arrival at healthcare facility, and door to reperfusion time in treatment of acute coronary syndrome (ACS) can be improved significantly if the patient or the relatives can record the 12-lead ECG at home and transmit it to the physician for prompt interpretation. The system (100) and the method (200) of the present disclosure thus facilitates the above by providing the wearable component (the wearable ECG acquisition unit (40)) that is simple and user friendly to allow the user or his relatives to perform recording of the 12-lead ECG at home without resorting to a skilled physician (since the proposed torso only positions are easily identifiable to lay person and are less cumbersome than the conventional placement positions) and then immediately transmit it to the nearest healthcare provider for interpretation to receive immediate medication therefrom. This reduction in time from onset of symptom to healthcare facility, and reduction in door to reperfusion time significantly helps in offering immediate treatment to the user on the cardiac condition thereof during early hours without resorting to any specialized medical facility. The system (100) and the method (200) can be deployed to facilitate early diagnosis of the cardiac, pulmonary disease and abnormalities related thereto, and is specifically useful in early and accurate diagnosis of heart attacks and ACS for early identification and initiation of treatment thereof during early hours of the onset of such an event even before the user actually reaches the healthcare facility. The system (100) and the method (200) also finds use in culprit artery localization when used during stress testing by interpretation of the 12-lead ECG (torso ECG) recorded. The system (100) allows the patient or wearer to conduct low-cost, comprehensive and real-time monitoring of their cardiac condition by the healthcare provider remotely. Data can be recorded several times a day to provide a relatively comprehensive data set facilitating the healthcare provider to remotely view the data set and observe trends in the data, which may be indicative of a medical condition. The present invention thus allows the wearer to make measurements with no discomfort thereto such that the measurements (of ECG signals) are made at the wearer or the patient's home or work, rather than in the medical facility located far away from the user. The system (100) is advantageous for both the patients and the athletes because the wearable ECG acquisition unit (40) can collect high quality 12-lead ECG and other physiological data (by deploying other sensors) while the wearer engages in activities of daily living. Thus, the system (100) and the method (200) of the present disclosure saves many lives and also avoids unnecessary hospital visits by making the monitoring of the cardiovascular health of the user wirelessly and remotely. The system (100) and the method (200) is applicable to the 12-lead ECG monitoring in wards, community health service areas or homes.

The foregoing objects of the invention are accomplished, and the problems and shortcomings associated with prior art techniques and approaches are overcome by the proposed invention described in the present embodiment. The foregoing descriptive matter is to be interpreted merely as an illustration of the concept of the present disclosure and it is in no way to be construed as a limitation. Detailed descriptions of the preferred embodiment are provided herein; however, it is to be understood that the proposed invention may be embodied in various forms. Therefore, specific details disclosed herein are not to be interpreted as limiting, but rather as a basis for the claims and as a representative basis for teaching one skilled in the art to employ the proposed invention in virtually any appropriately detailed system, structure, or matter. The embodiments of the invention as described above are non-limiting, and the processes disclosed herein will suggest further modification and alterations to those skilled in the art. Such further modifications and alterations may be made without departing from the scope of the invention.

Technical Advantages and Economic Significance

The technical advantages and economic significance of the system (100) and the method (200) of the present disclosure include but are not limited to:
a) universal and economical;
b) provides the wearable ECG acquisition unit (40) which is wearable, portable and user-friendly;
c) facilitates efficient real time and wireless remote monitoring of patient cardiac activity;
d) facilitates ECG monitoring during various recreational activities akin to home stress testing monitored remotely;
e) facilitates remote ECG monitoring during occupational activities thus providing useful inputs on vocational guidance;
f) facilitates accurate and precise culprit artery localization in event of ischemia since the system (100) and the method (200) uses the 12-lead electrode configuration for recording ECG during stress testing;
g) easy to deploy and execute without specialized training;
h) highly reliable, user friendly and responsive in diagnosis of cardiac abnormalities, including coronary artery disease, acute coronary syndromes, cardiomyopathy and like, and specifically useful for accurate home and remote diagnosis of heart attacks; and
i) universality in electrode placement positions essentially records same ECG across the ECG modalities, thereby allowing extrapolation across different monitoring facilities such as ICU ECG monitoring, ECG monitoring during surgery or interventional cardiac procedures, telemetry monitoring, stress testing and ambulatory ECG monitoring.

I claim:

1. A system (100) for 12-lead ECG recording and wireless remote monitoring, the system (100) being capable of recording a 12-lead ECG that is equivalent to a standard 12-lead ECG recorded using a conventional 12-lead ECG system, the system (100) comprising:
a wearable ECG acquisition unit (40) being adapted to acquire the 12-lead ECG when worn by a user around torso region thereof, the wearable ECG acquisition unit (40) comprising of:
a support substrate,
a first component being an electrode sensor assembly (10) preconfigured and fixedly positioned across the support substrate for being adhered at positions on the torso region of the user to collectively acquire 12-lead ECG signals therefrom when the wearable ECG acquisition unit (40) is worn by the user, the electrode sensor assembly (10) comprising,
a) six precordial electrode sensors (V1-V6) provided for being adhered to chest region of the user for acquiring precordial ECG signals therefrom, the six precordial electrode sensors (V1-V6) preconfigured at fixed predetermined positions across the support substrate for adhering to the chest region of the user at positions such that V1 and V2 gets disposed to lie on fourth intercostal space on opposite side of sternum and equidistant therefrom, V4 gets disposed to lie on fifth intercostal space at mid-clavicular line of the user, V3 gets disposed to lie midway between placement position of V2 and V4 whereas V5 and V6 gets disposed to lie on the fifth intercostal space at anterior-axillary line and mid-axillary line respectively upon wearing of the wearable ECG acquisition unit (40) by the user, wherein, positions of the V4, V5 and V6 generally define a horizontal plane, and
b) four limb electrode sensors (RA, LA, RL, LL) provided for being adhered to the chest and abdominal region of the user for acquiring ECG signals therefrom, the four limb electrode sensors (RA, LA, RL, LL) preconfigured at fixed predetermined positions across the support substrate for adhering to the chest and abdominal region of the user at positions such that RA and LA gets disposed to lie exactly in or adjacent to right and left delto-pectoral groove of the user below lateral end of clavicle respectively whereas RL and LL gets disposed to lie at a distance ranging from 1-3 inches above and left-lateral to umbilicus of the user at 12 o'clock and 3 o'clock position respectively upon wearing of the wearable ECG acquisition unit (40) by the user, and
a second component being a signal processing unit (20) electrically coupled to the first component for receiving the 12-lead ECG signals therefrom, the signal processing unit (20) being capable of performing processing and digitization of the 12-lead ECG signals to generate the 12-lead ECG therefrom indicative of cardiovascular health of the user, wherein the signal processing unit (20) being adapted with capability of wireless transmission of the 12-lead ECG of the user to facilitate wireless remote monitoring cardiac condition thereof;
a plurality of communication device (80) coupled over a communication network (60) to the second component of the wearable ECG acquisition unit (40) for receiving the 12-lead ECG generated and transmitted wirelessly therefrom, each of the plurality of communication device (80) represents a computing device configured with a web application for enabling real-time interaction and remote exchange of information therebetween over the communication network (60) thereby facilitating wireless remote monitoring of the cardiovascular health of the user by a healthcare provider, wherein, the user and the healthcare provider interacts using the web application executing on the communication device (80) independently coupled thereto; and
a communication module operatively coupled to the second component and to each of the plurality of communication device (80) for establishing the communication network (60) therebetween and therewithin to enable effective real-time monitoring and management of the cardiovascular health of the user by the healthcare provider,
wherein, adherence of the four limb electrode sensors (RA, LA, RL, LL) at said positions on the chest and the abdominal region of the user imparts portability and wearability whilst facilitating acquisition of the 12-lead ECG across ECG modalities without alteration in placement/adherence positions of the electrode sensor assembly (10) thereby imparting universality with respect thereto and enabling uniform acquisition of the 12-lead ECG across ECG modalities allowing extrapolation of ECG changes thereacross, wherein, the ECG modalities includes recording of resting, ambulatory and stress ECG and continuous monitoring of ECG during ongoing surgical procedures in ICU and telemetry.

2. The system (100) as claimed in claim 1, wherein the wearable ECG acquisition unit (40) is fabricated in at least one form selected from a group consisting of a vest, a jacket, a pad, a garment, a pador band, a shirt, a strip, a strap, a band, a belt, a plate, a patch, a mask, a harness and combinations thereof.

3. The system (100) as claimed in claim 1, wherein the wearable ECG acquisition unit (40) further comprises of a GPS module to provide location information of the user to the healthcare provider.

4. The system (100) as claimed in claim 1, wherein the communication module comprises of anyone of a wired and a wireless communication network module, and preferably includes a wireless communication network module for establishing a wireless communication network within the system (100).

5. A method (200) for 12-lead ECG recording and wireless remote monitoring, the method (200) being capable of recording a 12-lead ECG that is equivalent to a standard 12-lead ECG recorded using a conventional 12-lead ECG system, the method (200) comprising the steps of:
providing a system for 12-lead ECG recording and wireless remote monitoring, the system (100) comprising:
a wearable ECG acquisition unit (40) comprising of a support substrate, a first component being an electrode sensor assembly (10) including six precordial electrode sensors (V1-V6) and four limb electrode sensors (RA, LA, RL, LL) preconfigured and fixedly positioned across the support substrate for adhering at positions on torso region of user to collectively acquire 12-lead ECG signals therefrom, and a second component being a signal processing unit (20) electrically coupled to the first component for receiving the 12-lead ECG signals to generate the 12-lead ECG therefrom when the wearable ECG acquisition unit (40) is worn by the user, a plurality of communication device (80) coupled to the second component over a communication network (60) for receiving the 12-lead ECG transmitted wirelessly therefrom, wherein each of the plurality of communication device (80) represents a computing device configured with a web application to facilitate interaction and real-time exchange of data therebetween, and a communication module operatively coupled to the system (100) for establishing the communication network (60) within components/parts of the system (100) thereby facilitating wireless remote monitoring and management of cardiovascular health of the user by a healthcare provider;
wearing of the wearable ECG acquisition unit (40) by the user around torso region thereof facilitating torsional placement of the electrode sensor assembly (10) such that amongst the six precordial electrode sensors (V1-V6), V1 and V2 gets disposed to lie on opposite side of sternum equidistant therefrom and on fourth intercostal space, V4 gets disposed to lie on fifth intercostal space at mid-clavicular line, V3 gets disposed to lie midway between V2 and V4, V5 and V6 gets disposed to lie on the fifth intercostal space at anterior-axillary line and mid-axillary line respectively, whereas amongst the four limb electrode sensors (RA, LA, RL, LL), RA and LA gets disposed to lie within or adjacent to right and left delto-pectoral groove respectively below lateral end of clavicle, and RL and LL gets disposed to lie at a distance ranging from 1-3 inches above and left lateral to umbilicus of the user at 12 o'clock and 3 o'clock position respectively to acquire ECG signals therefrom;
sensing and acquisition of precordial ECG signals and ECG signals by the six precordial electrode sensors (V1-V6) and the four limb electrode sensors (RA, LA, RL, LL) respectively thereby collectively facilitating acquisition of 12-lead ECG signals from corresponding positions on chest and abdominal region of the user;
transmission of the 12-lead ECG signals by the electrode sensor assembly (10) to the signal processing unit (20) electrically coupled thereto;
processing of the 12-lead ECG signals to generate the 12-lead ECG therefrom by the signal processing unit (20), wherein the 12-lead ECG indicates cardiovascular health of the user;
transmission of generated 12-lead ECG to a user communication device by the signal processing unit (20) coupled thereto over a communication network (60), and preferably a wireless communication network (60);
routing of the 12-lead ECG to a healthcare provider communication device located remote to the user by the web application executing on the user communication device over the communication network (60), wherein the healthcare provider remotely views and analyzes user's data via a healthcare provider interface of the web application executing on the healthcare provider communication device to provide expert comments to the user; and
receiving remote medical assistance and medication by the user via the web application executing on the user communication device from the healthcare provider communication device coupled thereto over the communication network (60) thereby facilitating effective real-time monitoring and management of the cardiovascular health of the user by the healthcare provider,
wherein, acquisition of the 12-lead ECG across ECG modalities is facilitated without alteration in placement/adherence positions of the electrode sensor assembly (10) thereby imparting universality with respect thereto and enabling uniform acquisition of the 12-lead ECG across the ECG modalities allowing extrapolation of ECG changes thereacross, wherein, the ECG modalities includes recording of resting, ambulatory and stress ECG and continuous monitoring of ECG during ongoing surgical procedures in ICU and telemetry.

6. The method (200) as claimed in claim 5, comprising a step of acquisition of location signals of the user by a GPS module operatively coupled to the wearable ECG acquisition unit (40) to additionally provide location information of the user to the healthcare provider thereby facilitating reception of immediate medical assistance in an unforeseen event.
